(19) United States
(12) Patent Application Publication
(10) Pub. No.: US 2006/0029947 A1
(43) Pub. Date: Feb. 9, 2006

(54) Combinatorial protein library screening by periplasmic expression

(75) Inventors: George Georgiou, Austin, TX (US); Ki Jun Jeong, Austin, TX (US); Brent L. Iverson, Austin, TX (US)

Correspondence Address: FULBRIGHT & JAWORSKI L.L.P., 600 CONGRESS AVE., SUITE 2400, AUSTIN, TX, 78701, US

(73) Assignee: Board of Regents, The University of Texas System

(21) Appl. No.: 11/084,055

(22) Filed: Mar. 18, 2005

Related U.S. Application Data
(60) Provisional application No. 60/554,260, filed on Mar. 18, 2004.

(51) Int. Cl. C40B 40/02; C12Q 1/68; G01N 33/53; C12P 21/06; C12N 15/74

(52) U.S. Cl. 435/6; 435/7.1; 435/69.1; 435/252.33; 435/488

(57) ABSTRACT

The invention overcomes the deficiencies of the prior art by providing a rapid approach for isolating binding proteins capable of binding small molecules and peptides. In the technique, libraries of candidate binding proteins, such as antibody sequences, may be expressed in the periplasm of gram negative bacteria with at least one target ligand. In clones expressing recombinant polypeptides with affinity for the ligand, the ligand becomes bound and retained by the cell even after removal of the outer membrane, allowing the cell to be isolated from cells not expressing a binding polypeptide with affinity for the target ligand. The target ligand may be detected in numerous ways, including use of direct fluorescence or secondary antibodies that are fluorescently labeled, allowing use of efficient screening techniques such as fluorescence activated cell sorting (FACS). The approach is more rapid and robust than prior art methods and avoids problems associated with the outer surface-expression of ligand fusion proteins employed with phage display.

This application claims the priority of U.S. Provisional Patent Application Ser. No. 60/554,260, filed Mar. 18, 2004, the entire disclosure of which is specifically incorporated herein by reference.

The government may own rights in the present invention pursuant to the U.S. Army ARO MUR1 program; the Texas Consortium for Development of Biological Sensors; U.S. Department of Defense TransTexas BW Defense Initiative Grant No. DAA21-93C-0101 and in connection with contract number DADD17-01-D-0001 with the U.S. Army Research Laboratory.

### BACKGROUND OF THE INVENTION

1. Field of the Invention

The present invention relates generally to the field of protein engineering. More particularly, it concerns improved methods for the screening of combinatorial libraries to allow isolation of ligand binding polypeptides.

2. Description of Related Art

The isolation of polypeptides that either bind to ligands with high affinity and specificity or catalyze the enzymatic conversion of a reactant (substrate) into a desired product is a key process in biotechnology. Ligand-binding polypeptides, including proteins and enzymes with a desired substrate specificity can be isolated from large libraries of mutants, provided that a suitable screening method is available. Small protein libraries composed of 10³-10⁵ distinct mutants can be screened by first growing each clone separately and then using a conventional assay for detecting clones that exhibit specific binding. For example, individual clones expressing different protein mutants can be grown in microtiter well plates or separate colonies on semisolid media such as agar plates. To detect binding the cells are lysed to release the proteins and the lysates are transferred to nylon filters, which are then probed using radiolabeled or fluorescently labeled ligands (DeWildt et al. 2000). However, even with robotic automation and digital image systems for detecting binding in high density arrays, it is not feasible to screen large libraries consisting of tens of millions or billions of clones. The screening of libraries of that size is required for the de novo isolation of enzymes or protein binders that have affinities in the subnanomolar range.

The screening of very large protein libraries has been accomplished by a variety of techniques that rely on the display of proteins on the surface of viruses or cells (Ladner et al. 1993). The underlying premise of display technologies is that proteins engineered to be anchored on the external surface of biological particles (i.e., cells or viruses) are directly accessible for binding to ligands without the need for lysing the cells. Viruses or cells displaying proteins with affinity for a ligand can be isolated in a variety of ways including sequential adsorption/desorption form immobilized ligand, by magnetic separations or by flow cytometry (Ladner et al. 1993, U.S. Pat. No. 5,223,409, Ladner et al. 1998, U.S. Pat. No. 5,837,500, Georgiou et al. 1997, Shusta et al. 1999).

The most widely used display technology for protein library screening applications is phage display. Phage display is a well-established and powerful technique for the discovery of proteins that bind to specific ligands and for the engineering of binding affinity and specificity (Rodi and Makowski, 1999). In phage display, a gene of interest is fused in-frame to phage genes encoding surface-exposed proteins, most commonly pIII. The gene fusions are translated into chimeric proteins in which the two domains fold independently. Phage displaying a protein with binding affinity for a ligand can be readily enriched by selective adsorption onto immobilized ligand, a process known as “panning”. The bound phage is desorbed from the surface, usually by acid elution, and amplified through infection of E. Coli cells. Usually, 4-6 rounds of panning and amplification are sufficient to select for phage displaying specific polypeptides, even from very large libraries with diversities up to 10¹⁰. Several variations of phage display for the rapid enrichment of clones displaying tightly binding polypeptides have been developed (Duenas and Borrebaeck, 1994; Malmborg et al., 1996; Kjaer et al., 1998; Burioni et al., 1998; Levitan, 1998; Mutuberria et al., 1999; Johns et al., 2000).

One of the most significant applications of phage display technology has been the isolation of high affinity antibodies (Dall'Acqua and Carter, 1998; Hudson et al., 1998; Hoogenboom et al., 1998; Maynard and Georgiou, 2000). Very large and structurally diverse libraries of scFv or FAB fragments have been constructed and have been used successfully for the in vitro isolation of antibodies to a multitude of both synthetic and natural antigens (Griffiths et al., 1994; Vaughan et al., 1996; Sheets et al., 1998; Pini et al., 1998; de Haard et al., 1999; Knappik et al., 2000; Sblattero and Bradbury, 2000). Antibody fragments with improved affinity or specificity can be isolated from libraries in which a chosen antibody had been subjected to mutagenesis of either the CDRs or of the entire gene CDRs (Hawkins et al., 1992; Low et al., 1996; Thompson et al., 1996; Chowdhury and Pastan, 1999). Finally, the expression characteristics of scFv, notorious for their poor solubility, have also been improved by phage display of mutant libraries (Deng et al., 1994; Coia et al., 1997).

However, several spectacular successes notwithstanding, the screening of phage-displayed libraries can be complicated by a number of factors. First, phage display imposes minimal selection for proper expression in bacteria by virtue of the low expression levels of antibody fragment gene III fusion necessary to allow phage assembly and yet sustain cell growth (Krebber et al., 1996, 1997). As a result, the clones isolated after several rounds of panning are frequently difficult to produce on a preparative scale in E. coli. Second, although phage displayed proteins may bind a ligand, in some cases their un-fused soluble counterparts may not (Griep et al., 1999). Third, the isolation of ligand-binding proteins and more specifically antibodies having high binding affinities can be complicated by avidity effects by virtue of the need for gene III protein to be present at around 5 copies per virion to complete phage assembly. Even with systems that result in predominantly monovalent protein display, there is nearly always a small fraction of clones that contain multiple copies of the protein. Such clones bind to the immobilized surface more tightly and are enriched relative to monovalent phage with higher affinities (Deng et al., 1995; MacKenzie et al., 1996, 1998). Fourth, theoretical analysis aside (Levitan, 1998), panning is still a “black box” process in that the effects of experimental conditions, for example the stringency of washing steps to remove weakly or non-specifically bound phage, can only be determined by trial and error based on the final outcome of the experiment. Finally, even though pIII and to a lesser extent the other proteins of the phage coat are generally tolerant to the fusion of heterologous polypeptides, the need to be incorporated into the phage biogenesis process imposes biological constraints that can limit library diversity. Therefore, there is a great need in the art for techniques capable of overcoming these limitations.

Protein libraries have also been displayed on the surface of bacteria, fungi, or higher cells. Cell displayed libraries are typically screened by flow cytometry (Georgiou et al. 1997, Daugherty et al. 2000). However, just as in phage display, the protein has to be engineered for expression on the outer cell surface. This imposes several potential limitations. For example, the requirement for display of the protein on the surface of a cell imposes biological constraints that limit the diversity of the proteins and protein mutants that can be screened. Also, complex proteins consisting of several polypeptide chains cannot be readily displayed on the surface of bacteria, filamentous phages or yeast. As such, there is a great need in the art for technology which circumvents all the above limitations and provides an entirety novel means for the screening of very large polypeptide libraries.

### SUMMARY OF THE INVENTION

In one aspect, the invention provides a method of obtaining a bacterium comprising a nucleic acid sequence encoding a binding polypeptide having specific affinity for a target ligand comprising the steps of: (a) providing a Gram negative bacterium comprising an inner membrane, an outer membrane and a periplasm; the bacterium comprising a nucleic acid sequence encoding a candidate binding polypeptide comprising an inner membrane anchor polypeptide; wherein the bacterium further comprises a nucleic acid sequence encoding a target ligand and wherein the target ligand is exported to the periplasm; (b) allowing the target ligand to bind to the candidate binding polypeptide in the periplasm; (c) removing unbound target ligand from the periplasm; and (d) selecting the bacterium based on the presence of the target ligand bound to the candidate binding polypeptide. Such a target ligand may comprise, for example, a complete protein as well antigenic portions thereof. The method may be further defined as a method of obtaining a nucleic acid sequence encoding a binding polypeptide having a specific affinity for a target ligand, the method further comprising the step of: (d) cloning the nucleic acid sequence encoding a candidate binding polypeptide from the bacterium.

In one embodiment of the method, selecting the bacterium comprises use of a second binding polypeptide having specific affinity for the target ligand to label the target ligand bound to the candidate binding polypeptide. The second binding polypeptide may be an antibody or fragment thereof and may be fluorescently labeled. Selecting the bacterium comprises use of at least a third binding polypeptide having specific affinity for the target ligand and/or the second binding polypeptide to label the bacterium. The target ligand may be fused to a detectable label, including an antigen or GFP. The target ligand may be further defined as fused to a cytoplasmic degradation signal, including SsrA. The Gram negative bacterium may be, for example, an E. coli bacterium.

In certain embodiments of the invention, step (a) is further defined as comprising providing a population of Gram negative bacteria. The population of bacteria may be defined as collectively expressing nucleic acid sequences encoding a plurality of candidate binding polypeptides. The population of bacteria may also be further defined as collectively expressing nucleic acid sequences encoding a plurality of target ligands. The population of bacteria may express a single target ligand. In the method, about two to six rounds of selecting may be carried out to obtain the bacterium from the population. A bacterium selected may be viable or non-viable. The method may comprise cloning using amplification of the nucleic acid sequence. The candidate binding polypeptide may be a fusion polypeptide and/or an antibody or fragment thereof, including a scAb, Fab or scFv and an enzyme. The target ligand may be selected from the group consisting of a peptide, a polypeptide, an enzyme, a nucleic acid and a small molecule. The nucleic acid encoding a candidate binding polypeptide may be flanked by known PCR primer sites.

In one embodiment of the invention, step (c) comprises permeabilizing and/or removing the outer membrane. Permeabilizing and/or removing the outer membrane may comprise, for example, a method selected from the group consisting of: treatment with hyperosmotic conditions, treatment with physical stress, infecting the bacterium with a phage, treatment with lysozyme, treatment with EDTA, treatment with a digestive enzyme and treatment with a chemical that disrupts the outer membrane, including combinations thereof, as well as physical, chemical and enzyme treatments. The bacterium may also comprise a mutation conferring increased permeability of the outer membrane. The bacterium may be grown at a sub-physiological temperature, including about 25° C.

In a method of the invention, the target ligand and the candidate binding polypeptide may be reversibly or irreversibly bound. The target ligand may be operably linked to a leader sequence capable of directing the export of the target ligand to the periplasm, for example, an ssTorA leader peptide. The inner membrane anchor polypeptide may comprise a transmembrane protein or fragment thereof, including a sequence selected from the group consisting of: the first two amino acids encoded by the E. coli NlpA gene, the first six amino acids encoded by the E. coli NlpA gene, the gene III protein of filamentous phage or a fragment thereof, an inner membrane lipoprotein or fragment thereof. The inner membrane anchor polypeptide may be fused to the candidate binding polypeptide via an N- or C-terminus. In certain embodiments, the inner membrane anchor polypeptide may comprise an inner membrane lipoprotein or fragment thereof selected from the group consisting of: AraH, MglC, MalF, MalG, Mal C, MalD, RbsC, RbsC, ArtM, ArtQ, GlnP, ProW, HisM, HisQ, LivH, LivM, LivA, Liv E,Dpp B, DppC, OppB,AmiC, AmiD, BtuC, FhuB, FecC, FecD,FecR, FepD, NikB, NikC, CysT, CysW, UgpA, UgpE, PstA, PstC, PotB, PotC,PotH, PotI, ModB, NosY, PhnM, LacY, SecY, TolC, DsbB, DsbD, TonB, TatC, CheY, TraB, Exb D, ExbB and Aas.

In another aspect, the invention provides a method of obtaining a bacterium comprising a nucleic acid sequence encoding a binding polypeptide having specific affinity for a target ligand comprising the steps of: (a) providing a Gram negative bacterium comprising an inner membrane, an outer membrane and a periplasm; the bacterium comprising a nucleic acid sequence encoding a candidate binding polypeptide, wherein the candidate binding polypeptide is anchored to the outer side of the inner membrane with an inner membrane anchor polypeptide; wherein the bacterium further comprises a nucleic acid sequence encoding a target ligand, wherein the target ligand is exported to the periplasm; (b) allowing the target ligand to bind to the candidate binding polypeptide; (c) removing the outer membrane of the bacterium; and (c) selecting the bacterium based on the presence of the target ligand bound to the candidate binding polypeptide on the outer side of the inner membrane.

In yet another aspect, the invention provides a method of obtaining a bacterium comprising a nucleic acid sequence encoding a binding polypeptide having specific affinity for a target ligand comprising the steps of: (a) providing a population of Gram negative bacteria the members of which comprise an inner membrane, an outer membrane and a periplasm; the population collectively comprising nucleic acid sequences encoding plurality of candidate binding polypeptides, wherein the candidate binding polypeptides are anchored to the outer side of the inner membrane of the bacteria; wherein the bacteria further comprise nucleic acid sequences encoding a target ligand, wherein the target ligand is exported to the periplasm; (b) allowing the target ligand to bind to the candidate binding protein in the periplasm; (c) removing the outer membrane of the bacterium; and (d) selecting the bacterium from the population based on the presence of the target ligand bound to the candidate binding polypeptide on the outer side of the inner membrane. In the method, step (d) may be further defined as selecting a subpopulation of bacteria comprising the target ligand bound to the candidate binding polypeptide. Step (d) may also comprise fluorescently labeling the target ligand followed by fluorescence activated cell sorting (FACS).

In still yet another aspect, the invention provides a method of obtaining a bacterium comprising a nucleic acid sequence encoding a binding polypeptide having specific affinity for a target ligand comprising the steps of: (a) providing a Gram negative bacterium comprising an inner membrane, an outer membrane and a periplasm; the bacterium comprising a nucleic acid sequence encoding a candidate binding polypeptide, wherein the candidate binding polypeptide is anchored to the outer side of the inner membrane; wherein the bacterium further comprises a nucleic acid sequence encoding a fusion polypeptide comprising a target ligand, a periplasmic export signal, a fluorescent label and a cytoplasmic degradation signal; (b) allowing the target ligand to bind to the candidate binding polypeptide; (c) removing the outer membrane of the bacterium; and (d) selecting the bacterium based on the presence of the target ligand bound to the candidate binding polypeptide on the outer side of the inner membrane using fluorescence activated cell sorting (FACS). In certain embodiments, the periplasmic export signal may be TorA and/or the cytoplasmic degradation signal may be SsrA. In one embodiment, the fluorescent label is GFP. In the method, the fusion polypeptide may comprise the following components from the N-terminus to C-terminus: a periplasmic export signal, a target ligand, a fluorescent label and a cytoplasmic degradation signal.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-C: Selective identification of Antigen targets with anchored periplasmic expression. The anchored expressed scFvs in E. coli represented as indicated. Shows scFvs expressed that bind small molecules, (FIG. 1A) digoxigenin-Bodipy FL, (FIG. 1B) methamphetamine-FL; or ScFvs expressed that bind peptides (FIG. 1C) e.g., peptide 18aa.

FIG. 2A-B: Detection of ScFvs on the Surface of Spheroplasts. Anchored expressed scFvs in E. coli represented as indicated. ScFvs expressed were capable of binding large antigens, e.g., PA-Cy5 (83 kD), Phycoerythrin-digoxigenin (240 kD). Provides evidence that scFvs expressed via APEx are accessible to large proteins.

FIG. 3A-B: Detection of ScFvs for Larger Target Antigen conjugated fluorophores.

FIG. 4: Maturation of methamphetamine binding scFv for Meth-FL probe.

FIG. 5: Analysis of clone designated mutant 9 with higher mean FL signal than the parent anti-methamphetamine scFv. The scFvs expressed via anchored periplasmic expression are as indicated.

FIG. 6: A schematic diagram showing the principle of Anchored Periplasmic Expression (APEx) for the flow cytometry based isolation of high affinity antibody fragments.

FIG. 7: Examples of targets visualized by periplasmic expression. (FIG. 7A) Fluorescence distribution of ABLEC™ cells expressing PA specific (14B7) and digoxigenin specific (Dig) scFv and labeled with 200 nM Bodipy™ conjugated fluorescent antigens. Histograms represent the mean fluorescence intensity of 10,000 E. Coli events. (FIG. 7B) Histograms of cells expressing 14B7 or Dig scFv labeled with 200 nM of the 240 kDa digoxigenin-phycoerythrin conjugate.

FIG. 8: Analysis of anti-PA antibody fragments selected using APEx (FIG. 8A) Signal Plasmon Resonance (SPR) analysis of anti-PA scAb binding to PA. (FIG. 8B) Table of affinity data acquired by SPR. (FIG. 8C) FC Histogram of anti-PA scFv in pAPEx1 expressed in E. coli and labeled with 200 nM PA-Bodipy™ conjugate as compared with anti-methamphetamine (Meth) scFv negative control.

FIG. 9: N-Terminal vs. C-Terminal anchoring strategy comparison. (FIG. 9A) Anti-digoxigenin Dig scfv, anti-PA M18 scFv and anti-methamphetamine Meth scFv expressed as N-terminal fusions in the pAPEx1 vector in E. coli specifically label with 200 nM of their respective antigen. (FIG. 9B) C-terminal fusions of same scFv in pAK200 vector specifically labeled with 200 nM of their respective antigen.

FIG. 10: View from the top of the antibody binding pocket showing the conformation and amino acid substitutions in the 1H, M5, M6 and M18 sequences.

FIG. 11: Alignment of 14B7 scFv (SEQ ID NO:21) and M18 scFv (SEQ ID NO:23) sequences showing variable heavy and variable light chains and mutations made to improve binding affinity.

FIG. 12: The structure of: (FIG. 12A) the 7C2 antigen peptide fused for GFP probe expression (pT7C2GS30) and (FIG. 12B) the 7C2 scFv-APEx system (S, SfiI; X, XbaI; B, BamHI; H, HindIII).

FIG. 13: Flow-cytometry analysis of (FIG. 13A) GFP-peptide fusion alone (pT7C2GS30), (FIG. 13B) GFP-peptide co-expressed with 26-10 scFv-APEx (pT7C2GS30 & 26-10 scFv-APEx), (FIG. 13C) GFP without peptide fusion coexpressed with 7C2 anti-peptide scFv-APEx (pTGS30 & p7C2 scFv-APEx) (FIG. 13D) GFP-peptide coexpressed with 7C2 anti-peptide scFv-APEx (pT7C2GS30 & p7C2 scFv-APEx).

FIG. 14: Shows map of PA-domain 4 expression vector (FIG. 14A) and M18 scFv APEx expression vector (FIG. 14B).

FIG. 15: Shows FACS data for: only PA-Domain 4 expression (FIG. 15A), co-expression of PA-Domain IV and 26-10 scFv APEx (FIG. 15B) and co-expression of PA-Domain IV and M18 scFv APEx (FIG. 15C). Only panel (FIG. 15C) shows a positive FACS signal, verifying the detection of the endogenously expressed antigen-antibody pair.

FIG. 16: Sequence of PelB-PA-Domain4-FLAG tag construct. The DNA sequence (FIG. 16A). The amino acid sequence (FIG. 16B). Italic characters indicate the PelB leader peptide, bold characters indicate the PA-Domain 4, and underlined characters showed the FLAG tag.

FIG. 17: Flow-cytometry analysis of PA-Domain 4 alone (pB30PelBD4FL), PA-Domain 4 co-expressed with 26-10 scFv-APEx (pB30PelBD4FL & 26-10 scFv-APEx), and PA-Domain 4 coexpressed with M18 anti-peptide scFv-APEx (pB30PelBD4FL & pM18 scFv-APEx).

FIG. 18: Flow-cytometry analysis of wild type PA-Domain 4 co-expressed with M18 anti-peptide scFv-APEx (pB30PelBD4FL & pM18 scFv-APEx), PA-Domain 4 (Y681A) co-expressed with M18 anti-peptide scFv-APEx (pB30D4Y681 & pM18 scFv-APEx), and PA-Domain 4 (Y688A) co-expressed with M18 anti-peptide scFv-APEx (pB30D4Y688 & pM18 scFv-APEx).

FIG. 19: The structure of the one plasmid system for co-expression of pM18scFv-APEx and PelB-PA-Domain4-FLAG.

FIG. 20: Flow-cytometry analysis of (FIG. 20A) two plasmid system for co-expression Domain 4 (WT) and M18 scFv (pB30PelBD4FL and pM18 scFv APEx), (FIG. 20B) one plasmid system for co-expression Domain 4 (WT) and M18 scFv (pM18 scFv-D4), (FIG. 20C) two plasmid system for co-expression Domain 4 (Y688A) and M18 scFv (pB30D4-Y688A and pM18 scFv APEx), and (FIG. 20D) one plasmid system for co-expression Domain 4 (Y688A) and M18 scFv (pM18 scFv-D4Y688).

### DESCRIPTION OF ILLUSTRATIVE EMBODIMENTS

The invention overcomes the limitations of the prior art by providing novel methods for the isolation of binding polypeptides, including antibodies or antibody fragments, that recognize specific molecular targets. In the technique, libraries of candidate binding polypeptide mutants can be constructed and expressed in Gram negative bacteria together with one or more target ligands. Those binding polypeptides having affinity for the co-expressed target ligand may be selected based on the presence of the target ligand associated with the binding polypeptide anchored to the periplasmic face of the inner membrane. The mutant polypeptides can be anchored by their expression as fusion proteins with inner membrane proteins or fragments thereof.

The target ligand and candidate binding protein may be co-expressed and allowed to associate in the periplasm. Those candidate binding proteins having an affinity for the target ligand will specifically bind the target ligand and retain it within the periplasm, facilitating detection of the bacterium and isolation of a nucleic acid encoding the binding polypeptide based on the presence of the target ligand. The technique may be facilitated by removing the periplasmic (outer) membrane of the bacterium following by washing to remove unbound target ligand while retaining target ligand having a specific affinity for a given binding protein. As used herein, the term “specific affinity” refers to an association that is specific to a particular set of molecules and not general to, for example, all proteins within a cell. An example of specific affinity is the relationship between an antibody or fragment thereof and a given antigen.

The display of heterologous proteins on microbial scaffolds has attractive applications in many different areas including vaccine development, bioremediation and protein engineering. In Gram negative bacteria there have been display systems designed which by virtue of a N or C terminal chimera fusion, proteins are displayed to the cell surface. Although there have been many different strategies used to direct protein localization, including fusions to outer membrane proteins, lipoproteins, surface structural proteins and leader peptides, many share the same limitations. One limitation is the size of the protein which can be displayed. Many display scaffolds can only tolerate a few hundred amino acids, which significantly limits the scope of proteins which can be displayed. Also, display implies that the protein of interest is situated such that it can interact with its environment, yet the major limitation of many of these systems is that the architecture of the outer surface of gram negative bacteria and in particular the presence of lipopolysaccharide (LPS) molecules having steric limitations that inhibit the binding of externally added ligands. Another limitation arises from the requirement that the displayed protein is localized on the external surface of the outer membrane. For this purpose the polypeptide must first be secreted across the cytoplasmic membrane must assemble properly in the outer membrane.

A binding polypeptide may be any type of molecule of at least two amino acid residues capable of binding a given ligand. By binding it is meant that immunological interaction takes place. Biosynthetic limitations restrict the kinds of proteins that can be displayed in this fashion. For example, large polypeptides (e.g., alkaline phosphatase) cannot be displayed on the E. coli surface (Stathopoulos et al., 1996).

In accordance with the invention, the limitations of the prior techniques can be overcome by the display of proteins anchored to the outer surface of the inner membrane. It was demonstrated using the technique that, by utilizing conditions that permeabilize the outer membrane, E. coli expressing inner membrane anchored scFv antibodies (approx. 30 kDa in size) can be labeled with a target antigen conjugated, for example, to a fluorophore and can subsequently be used to sort protein libraries utilizing flow cytometry for isolation of gain of function mutants. The co-expression of target ligands and candidate binding polypeptides in particular constitutes a robust selection technique provided by the invention.

Candidate binding polypeptides may be anchored to the bacterial inner membrane using selected anchor polypeptides. As used herein, an inner membrane anchor polypeptide refers to any peptide sequence capable of binding a candidate binding polypeptide to the outer face of the inner membrane of a Gram negative bacterium. The inner membrane anchor polypeptide need not permanently bind to the inner membrane, but the association is sufficiently strong to allow removal of the outer membrane while maintaining candidate binding protein anchored to the outer face of the inner membrane. Inner membrane proteins and other sequences suitable for use as inner membrane anchor polypeptides are discussed in detail herein below.

Following disruption of the outer bacterial membrane, which is well known to those of skill in the art and may comprise, for example, use of Tris-EDTA-lysozyme, labeled antigens with sizes up to at least 240 kDa can be detected. With fluorescent labeling, cells may be isolated by flow cytometry and the DNA of isolated clones rescued by PCR. In one embodiment of the invention, target molecules are labeled with fluorescent dyes. Thus, bacterial clones expressing polypeptides that recognize the target molecule bind to the fluorescently labeled target and in turn become fluorescent. The fluorescent bacteria expressing the desired binding proteins can then be enriched from the population using automated techniques such as flow cytometry.

Polypeptide libraries can be attached to the periplasmic face of the inner membrane of E. coli or other Gram negative bacteria via fusion to an inner membrane anchor polypeptide. One example of an anchor that can be used comprises the first six amino acids of the NlpA (New Lipoprotein A) gene of E. coli. However, other single transmembrane or polytropic membrane proteins or peptide sequences can also be used for anchoring purposes.

One benefit of the technique is that anchoring candidate binding polypeptides to the periplasmic face of the inner membrane allows the permeabilization and removal of the bacterial outer membrane, which would normally limit the accessibility of the polypeptides to labeled target molecules. The anchoring of the binding polypeptide to the periplasmic face of the membrane prevents it from being released from the cell when the outer membrane is compromised. The technique can thus be used for the isolation of large binding polypeptides and ligands, including antibodies and other binding proteins from combinatorial libraries. The technique not only provides a high signal-to-noise ratio, but also allows the isolation of polypeptide or antibody binders to very large antigen molecules. Because the method allows selection of targets of greater size, there is the potential for use in the selection of targets such as specific antigen markers expressed on cells including tumor cells such as melanoma or other specific types of tumor cells. Tumor specific antibodies have shown great promise in the treatment of cancer.

The periplasm comprises the space defined by the inner and outer membranes of a Gram-negative bacterium. In wild-type E. coli and other Gram negative bacteria, the outer membrane serves as a permeability barrier that severely restricts the diffusion of molecules greater than 600 Da into the periplasmic space (Decad and Nikado, 1976). Conditions that increase the permeability of the outer membrane, allowing larger molecules to diffuse in the periplasm, have two deleterious effects in terms of the ability to screen libraries: (a) the cell viability is affected to a significant degree and (b) the diffusion of molecules into the cell is accompanied by the diffusion of proteins and other macromolecules.

Target ligands may be expressed in the periplasm of bacteria in accordance with the invention using any of the many well known techniques in the art for doing so. Examples of such techniques that may be used are described in, for example, U.S. patent application Ser. No. 09/699,023, filed Oct. 27, 2000, the entire disclosure of which is specifically incorporated herein by reference. In certain embodiments of the invention, a target ligand may be exported to the periplasm using the Twin Arginine Translocation (TAT) pathway. Exemplary techniques for exporting polypeptides with the TAT pathway are described in, for example, in U.S. Patent Application Publication No. 2003/0219870, the disclosure of which is specifically incorporated herein by reference in its entirety. Techniques for the isolation of additional leader peptides for exporting polypeptides to the periplasm are also known in the art and are disclosed in, for example, U.S. Patent Application pub. No. 2003/0180937, the disclosure of which is specifically incorporated herein by reference in its entirety.

The inventors, by providing techniques for anchoring candidate binding polypeptides to the outer (periplasmic) side of the inner membrane with co-expression of target ligands allow use of fluorescent conjugates to detect target ligands that are bound to an anchored binding protein on the inner membrane. Therefore, in bacterial cells expressing recombinant polypeptides with affinity for the ligand that is expressed, the ligand bound to the binding protein can be detected, allowing the bacteria to be isolated from the rest of the library. Where fluorescent labeling of the target ligand is used, cells may efficiently be isolated by flow cytometry, for example, fluorescence activated cell sorting (FACS). The ligand may also be expressed as a fusion with a directly detectable marker, such as GFP or another visible marker, or an secondarily detectable agent such as an antigen. With this approach, existing libraries of expressed fusion proteins in bacteria can be easily tested for ligand binding without the need for subcloning into a phage or outer cell surface display systems.

I. Anchored Periplasmic Expression

Prior art methods of both phage display and bacterial cell surface display suffer from a limitation in that the protein is required, by definition, to be physically displayed on the outer surface of the vehicle used, to allow unlimited access to the targets (immobilized for phage or fluorescently conjugated ligands for flow cytometry) (U.S. Pat. No. 5,223,409, the disclosure of which is specifically incorporated herein by reference in its entirety). However, certain proteins are known to be poorly displayed on phage (Maenaka et al., 1996; Corey et al., 1993) and the toxic effects of outer cell surface display have been treated at length (Daugherty et al., 1999). Further, there is no lipopolysaccharide to interfere with binding on the inner membrane.

Herein, the inventors have described a technique in which binding proteins can be expressed on the periplasmic face of the inner membrane as fusion proteins yet be accessible to relatively large ligands that are also expressed in the bacterium. As used herein, the term “binding polypeptide” includes not only antibodies, but also fragments of antibodies, as well as any other peptides, including proteins potentially capable of binding a given target molecule. The antibody or other binding peptides may be expressed with the invention as fusion polypeptides with polypeptides capable of serving as anchors to the periplasmic face of the inner membrane. Such a technique may be termed “Anchored Periplasmic Expression” or “APEx”.

The periplasmic compartment is contained between the inner and outer membranes of Gram negative cells (see, e.g., Oliver, 1996). As a sub-cellular compartment, it is subject to variations in size, shape and content that accompany the growth and division of the cell. Within a framework of peptidoglycan heteroploymer is a dense milieu of periplasmic proteins and little water, lending a gel-like consistency to the compartment (Hobot et al., 1984; van Wielink and Duine, 1990). The peptidoglycan is polymerized to different extents depending on the proximity to the outer membrane, close-up it forms the murein sacculus that affords cell shape and resistance to osmotic lysis.

The outer membrane (see Nikaido, 1996) is composed of phospholipids, porin proteins and, extending into the medium, lipopolysaccharide (LPS). The molecular basis of outer membrane integrity resides with LPS ability to bind divalent cations (Mg2+ and Ca2+) and link each other electrostatically to form a highly ordered quasi-crystalline ordered “tiled roof” on the surface (Labischinski et al., 1985). The membrane forms a very strict permeability barrier allowing passage of molecules no greater than around 650 Da (Burman et al., 1972; Decad and Nikaido, 1976) via the porins. The large water filled porin channels are primarily responsible for allowing free passage of mono and disaccharides, ions and amino acids in to the periplasm compartment (Naeke, 1976; Nikaido and Nakae, 1979; Nikaido and Vaara, 1985). With such strict physiological regulation of access by molecules to the periplasm it may appear that only ligands at or below the 650 Da exclusion limit or analogues of normally permeant compounds would access the periplasm. However, the inventors have shown that ligands greater than 2000 Da in size can diffuse into the periplasm without disruption of the periplasmic membrane. Such diffusion can be aided by one or more treatments of a bacterial cell, thereby rendering the outer membrane more permeable, as is described herein below. Further, anchoring of binding proteins allows removal of the outer membrane to facilitate detection, eliminating any theoretical limitation on the size of molecules having access to anchored polypeptides or the ligands bound to the polypeptides.

II. Screening Candidate Molecules

The present invention provides methods for identifying molecules capable of binding a target ligand. The binding polypeptides screened may comprise large libraries of diverse candidate substances, or, alternatively, may comprise particular classes of compounds selected with an eye towards structural attributes that are believed to make them more likely to bind the target ligand. In one embodiment of the invention, the candidate binding polypeptide is an antibody, or a fragment or portion thereof. In other embodiments of the invention, the candidate molecule may be another binding polypeptide.

To identify a candidate molecule capable of binding a target ligand in accordance with the invention, one may carry out the steps of: providing a population of Gram negative bacterial cells comprising fusion proteins between candidate binding polypeptides and a sequence anchored to the periplasmic face of the inner membrane; the bacteria expressing at least a first target ligand capable of contacting the candidate binding polypeptide in the periplasm and identifying at least a first bacterium expressing a molecule capable of binding the target ligand.

In the aforementioned method, the binding between the anchored candidate binding protein and the target ligand will prevent diffusing out of the cell. In this way, molecules of the target ligand can be retained in the periplasm of the bacterium and detected. Alternatively, the periplasm can be removed, whereby the anchoring will cause retention of the bound candidate molecule. Labeling may then be used to isolate the cell expressing a binding polypeptide capable of binding the target ligand, and in this way, the gene encoding the binding polypeptide isolated. The molecule capable of binding the target ligand may then be produced in large quantities using in vivo or ex vivo expression methods, and then used for any desired application, for example, for diagnostic or therapeutic applications, as described below.

As used herein the term “candidate molecule” or “candidate binding polypeptide” refers to any molecule or polypeptide that may potentially have affinity with a target ligand. The candidate substance may be a protein or fragment thereof, including a small molecule such as synthetic molecule. The candidate molecule may, in one embodiment of the invention, comprise an antibody sequence or fragment thereof. Such sequences may be particularly designed for the likelihood that they will bind a target ligand.

Binding polypeptides or antibodies isolated in accordance with the invention also may help ascertain the structure of a target ligand. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen. On the other hand, one may simply acquire, from various commercial sources, small molecule libraries that are believed to meet the basic criteria for binding the target ligand. Such libraries could be provided by way of nucleic acids encoding the small molecules or bacteria expressing the molecules.

A. Cloning of Binding Polypeptide Coding Sequences

The binding affinity of an antibody or other binding polypeptide can, for example, be determined by the Scatchard analysis of Munson & Pollard (1980). After a bacterial cell is identified that produces molecules of the desired specificity, affinity, and/or activity, the corresponding coding sequence may be cloned. In this manner, DNA encoding the molecule can be isolated and sequenced using conventional procedures (e.g., by using oligonucleotide probes that are capable of binding specifically to genes encoding the antibody or binding protein).

Once isolated, the binding protein DNA may be placed into expression vectors, which can then transfected into host cells such as simian COS cells, Chinese hamster ovary (CHO) cells, or myeloma cells that do not otherwise produce immunoglobulin protein, to obtain the synthesis of binding protein in the recombinant host cells. The DNA also may be modified, for example, by substituting the coding sequence for human heavy and light chain constant domains in place of the homologous murine sequences (Morrison, et al., 1984), or by covalently joining to the immunoglobulin coding sequence all or part of the coding sequence for a non-immunoglobulin polypeptide. In that manner, “chimeric” or “hybrid” binding proteins are prepared that have the desired binding specificity.

Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for the target ligand and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

It will be understood by those of skill in the art that nucleic acids may be cloned from viable or inviable cells. In the case of inviable cells, for example, it may be desired to use amplification of the cloned DNA, for example, using PCR. This may also be carried out using viable cells either with or without further growth of cells.

B. Maximization of Protein Affinity for Ligands

In a natural immune response, antibody genes accumulate mutations at a high rate (somatic hypermutation). Some of the changes introduced will confer higher affinity, and B cells displaying high-affinity surface immunoglobulin. This natural process can be mimicked by employing the technique known as “chain shuffling” (Marks et al., 1992). In this method, the affinity of “primary” human antibodies obtained in accordance with the invention could be improved by sequentially replacing the heavy and light chain V region genes with repertoires of naturally occurring variants (repertoires) of V domain genes obtained from unimmunized donors. This technique allows the production of antibodies and antibody fragments with affinities in the nM range. A strategy for making very large antibody repertoires was described by Waterhouse et al., (1993), and the isolation of a high affinity human antibody directly from such large phage library was reported by Griffith et al., (1994). Gene shuffling also can be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by the phage display technique is replaced with a repertoire of human V domain genes, creating rodent-human chimeras. Selection of the antigen results in isolation of human variable regions capable of restoring a functional antigen-binding site, i.e. the epitope governs (imprints) the choice of partner. When the process is repeated in order to replace the remaining rodent V domain, a human antibody is obtained (see PCT patent application WO 93/06213, published Apr. 1, 1993). Unlike traditional humanization of rodent antibodies by CDR grafting, this technique provides completely human antibodies, which have no framework or CDR residues of rodent origin.

C. Detection Agents

In one embodiment of the invention, an antibody or binding protein is isolated which has affinity for a target ligand co-expressed in a host bacterial cell. By removal of the outer membrane of a Gram negative bacterium in accordance with the invention, detection reagents of potentially any size could be used to screen for bound target ligand. In the absence of removal of the periplasmic membrane, it will typically be preferable that such reagents are less that 50,000 Da in size in order to allow efficient diffusion across the bacterial periplasmic membrane.

Labeling of a bound ligand can be carried out, for example, by binding the ligand with at least one detectable agent to form a conjugate. For example, it is conventional to link or covalently bind or complex at least one detectable molecule or moiety. A “label” or “detectable label” is a compound and/or element that can be detected due to specific functional properties, and/or chemical characteristics, the use of which allows the reagent to which it is attached to be detected, and/or further quantified if desired. Examples of labels which could be used with the invention include, but are not limited to, enzymes, radiolabels, haptens, fluorescent labels, phosphorescent molecules, chemiluminescent molecules, chromophores, luminescent molecules, photoaffinity molecules, colored particles and ligands, such as biotin.

In one embodiment of the invention, a visually-detectable marker is used such that automated screening of cells for the label can be carried out. In particular, fluorescent labels are beneficial in that they allow use of flow cytometry for isolation of cells expressing a desired binding protein or antibody. Examples of agents that may be detected by visualization with an appropriate instrument are known in the art, as are methods for their attachment to a desired reagent (see, e.g., U.S. Pat. Nos. 5,021,236; 4,938,948; and 4,472,509, each incorporated herein by reference). Such agents can include paramagnetic ions; radioactive isotopes; fluorochromes; NMR-detectable substances and substances for X-ray imaging. Types of fluorescent labels that may be used with the invention will be well known to those of skill in the art and include, for example, Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Magnetic screening techniques are well known to those of skill in the art (see, for example, U.S. Pat. No. 4,988,618, U.S. Pat. No. 5,567,326 and U.S. Pat. No. 5,779,907). Examples of paramagnetic ions that could be used as labels in accordance with such techniques include ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III). Ions useful in other contexts include but are not limited to lanthanum (III), gold (III), lead (II), and especially bismuth (III).

Another type of detecting reagent contemplated in the present invention are those where the reagent is linked to a secondary binding molecule and/or to an enzyme (an enzyme tag) that will generate a colored product upon contact with a chromogenic substrate. Examples of such enzymes include urease, alkaline phosphatase, (horseradish) hydrogen peroxidase or glucose oxidase. In such instances, it will be desired that cells selected remain viable. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

It will also be understood that a target ligand may be expressed with a label. For example, the target ligand may be expressed as a fusion protein with a label such as GFP. Numerous antigens could also be fused to the target ligand to facilitate detection.

Molecules containing azido groups also may be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide-binding proteins in crude cell extracts (Owens & Haley, 1987; Atherton et al., 1985). The 2- and 8-azido nucleotides have also been used to map nucleotide-binding domains of purified proteins (Khatoon et al., 1989; King et al., 1989; and Dholakia et al., 1989) and may be used as ligand binding agents.

Labeling can be carried out by any of the techniques well known to those of skill in the art. For instance, ligands can be labeled by contacting the ligand with the desired label and a chemical oxidizing agent such as sodium hypochlorite, or an enzymatic oxidizing agent, such as lactoperoxidase. Similarly, a ligand exchange process could be used. Alternatively, direct labeling techniques may be used, e.g., by incubating the label, a reducing agent such as SNCl₂, a buffer solution such as sodium-potassium phthalate solution, and the ligand. Intermediary functional groups on the ligand could also be used, for example, to bind labels to a ligand in the presence of diethylenetriaminepentaacetic acid (DTPA) or ethylene diaminetetracetic acid (EDTA).

Other methods are also known in the art for the attachment or conjugation of a ligand to its conjugate moiety. Some attachment methods involve the use of an organic chelating agent such as diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the ligand (U.S. Pat. Nos. 4,472,509 and 4,938,948, each incorporated herein by reference). Ligands also may be reacted with an enzyme in the presence of a coupling agent such as glutaraldehyde or periodate. Conjugates with fluorescein markers can be prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

Once a ligand-binding polypeptide, such as an antibody, has been isolated in accordance with the invention, it may be desired to link the molecule to at least one agent to form a conjugate to enhance the utility of that molecule. For example, in order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Techniques for labeling such a molecule are known to those of skill in the art and have been described herein above.

Labeled binding proteins such as antibodies which have been prepared in accordance with the invention may also then be employed, for example, in immunodetection methods for binding, purifying, removing, quantifying and/or otherwise generally detecting biological components such as protein(s), polypeptide(s) or peptide(s). Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H and Ben-Zeev O, 1999; Gulbis B and Galand P, 1993; and De Jager R et al., 1993, each incorporated herein by reference. Such techniques include binding assays such as the various types of enzyme linked immunosorbent assays (ELISAs) and/or radioimmunoassays (RIA) known in the art.

The ligand-binding molecules, including antibodies, prepared in accordance with the present invention may also, for example, in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Abbondanzo et al., 1990).

III. Permeabilization of the Outer Membrane

In one embodiment of the invention, methods are employed for increasing the permeability of the outer membrane for labeling and detection of bound target ligand. This may include complete removal of the outer membrane. By “removal” it is meant the removal of at least a portion of the outer membrane, preferably removal of at least about 25% of the outer membrane surface, including at least about 50% or 75% of the outer membrane surface. This can allow screening access with detection reagents otherwise unable to cross the outer membrane. This will also facilitate removal of unbound target ligand to reduce background noise.

Certain classes of molecules, for example, hydrophobic antibiotics larger than the 650 Da exclusion limit, can diffuse through the bacterial outer membrane itself, independent of membrane porins (Farmer et al., 1999). The process may actually permeabilize the membrane on so doing (Jouenne and Junter, 1990). Such a mechanism has been adopted to selectively label the periplasmic loops of a cytoplasmic membrane protein in vivo with a polymyxin B nonapeptide (Wada et al., 1999). Also, certain long chain phosphate polymers (100 Pi) appear to bypass the normal molecular sieving activity of the outer membrane altogether (Rao and Torriani, 1988).

Conditions have been identified that lead to the permeation of compounds into the periplasm without loss of viability or release of the expressed proteins from the cells, but the invention may be carried out without maintenance of the outer membrane. By anchoring candidate binding polypeptides to the outer side of the inner (cytoplasmic) membrane using fusion polypeptides, the need for maintenance of the outer membrane (as a barrier to prevent the leakage of the biding protein from the cell) to detect bound target ligand is removed. As a result, cells expressing binding proteins anchored to the outer (periplasmic) face of the cytoplasmic membrane can be fluorescently labeled simply by incubating with a solution of a labeled compound having an affinity for the target ligand. It is understood that by “labeled” it is meant that the compound would be detectable but need not itself have a marker such as fluorescence. For example, the target ligand may be detected with a mouse antibody having affinity for the target ligand but not itself fluorescently labeled followed by a fluorescently labeled rabbit antibody having affinity for the mouse antibody. Such a scheme can be repeated in multiple layers with various different types of binding proteins.

The permeability of the outer membrane of different strains of bacterial hosts can vary widely. It has been shown previously that increased permeability due to OmpF overexpression was caused by the absence of a histone like protein resulting in a decrease in the amount of a negative regulatory mRNA for OmpF translation (Painbeni et al., 1997). Also, DNA replication and chromosomal segregation is known to rely on intimate contact of the replisome with the inner membrane, which itself contacts the outer membrane at numerous points. A preferred host for library screening applications is E. coli ABLEC strain, which additionally has mutations that reduce plasmid copy number.

The inventors have also noticed that treatments such as hyperosmotic shock can improve labeling significantly. It is known that many agents including, calcium ions (Bukau et al., 1985) and even Tris buffer (Irvin et al., 1981) alter the permeability of the outer-membrane. Further, the inventors found that phage infection stimulates the labeling process. Both the filamentous phage inner membrane protein pIII and the large multimeric outer membrane protein pIV can alter membrane permeability (Boeke et al., 1982) with mutants in pIV known to improve access to maltodextrins normally excluded (Marciano et al., 1999). Using the techniques of the invention, comprising a judicious combination of strain, salt and phage, a high degree of permeability was achieved (Daugherty et al., 1999). Cells comprising anchored binding polypeptides bound to target ligands that are directly or indirectly labeled can then be easily isolated from cells that express binding proteins without affinity for the target ligand using flow cytometry or other related techniques. However, it will typically be desired to use less disruptive techniques in order to maintain the viability of cells. EDTA and Lysozyme treatments may also be useful in this regard.

IV. Anchoring of Heterologous Polypeptides

In one embodiment of the invention, bacterial cells are provided expressing fusion polypeptides on the outer face of the inner membrane. Such a fusion polypeptide may comprise a fusion between a candidate binding polypeptide and a polypeptide serving as an anchor to the outer face of the inner membrane. It will be understood to those of skill in the art that additional polypeptide sequences may be added to the fusion polypeptide and not depart from the scope of the invention. One example of such a polypeptide is a linker polypeptide serving to link the anchor polypeptide and the candidate binding polypeptide. The general scheme behind the invention comprises the advantageous expression of a heterogeneous collection of candidate binding polypeptides.

Anchoring to the inner membrane may be achieved by use of the leader peptide and the first six amino acids of an inner membrane lipoprotein. One example of an inner membrane lipoprotein is NlpA (new lipoprotein A). The first six amino acid of NlpA can be used as an N terminal anchor for protein to be expressed to the inner membrane. NlpA was identified and characterized in Escherichia coli as a non-essential lipoprotein that exclusively localizes to the inner membrane (Yu, 1986; Yamaguchi, 1988).

As with all prokaryotic lipoproteins, NlpA is synthesized with a leader sequence that targets it for translocation across the inner membrane via the Sec pathway. Once the precursor protein is on the outer side of the inner membrane the cysteine residue of the mature lipoprotein forms a thioether bond with diacylglyceride. The signal peptide is then cleaved by signal peptidase II and the cysteine residue is aminoacylated (Pugsley, 1993). The resulting protein with its lipid modified cysteine on its N terminus can then either localize to the inner or outer membrane. It has been demonstrated that this localization is determined by the second amino acid residue of the mature lipoprotein (Yamaguchi, 1988). Aspartate at this position allows the protein to remain anchored via its N terminal lipid moiety to the inner membrane, whereas any other amino acid in the second position generally directs the lipoprotein to the outer membrane (Gennity and Inouye, 1992). This is accomplished by proteins LolA, LolB and the ATP dependant ABC transporter complex LolCDE (Yakushi, 2000, Masuda 2002). NlpA has aspartate as its second amino acid residue and therefore remains anchored within the inner membrane.

It has been reported that by changing amino acid 2 of lipoproteins to an Arginine (R) will target them to reside in the inner membrane (Yakushi, 1997). Therefore all lipoproteins in E. coli (and potentially other Gram negative bacteria) can be anchor sequences. All that is required is a signal sequence and an arginine at amino acid 2 position. This construct could be designed artificially using an artificial sec signal sequence followed by the sec cleavage region and coding for cysteine as amino acid 1 and arginine as amino acid 2 of the mature protein. Transmembrane proteins could also potentially be used as anchor sequences although this will require a larger fusion construct.

Examples of anchors that may find use with the invention include lipoproteins, such as Pullulanase of K. pneumoniae, which has the CDNSSS mature lipoprotein anchor, phage encoded celB, and E. coli acrE (envC). Examples of additional inner membrane proteins which can be used as protein anchors include: AraH, MglC, MalF, MalG, Mal C, MalD, RbsC, RbsC, ArtM, ArtQ, GlnP, ProW, HisM, H is Q, LivH, LivM, LivA, Liv E,Dpp B, DppC, OppB,AmiC, AmiD, BtuC, FhuB, FecC, FecD,FecR, FepD, NikB, NikC, CysT, CysW, UgpA, UgpE, PstA, PstC, PotB, PotC,PotH, PotI, ModB, NosY, PhnM, LacY, SecY, TolC, DsbB, DsbD, TonB, TatC, CheY, TraB, Exb D, ExbB and Aas. Further, a single transmembrane loop of any cytoplasmic protein can be used as a membrane anchor.

The preparation of diverse populations of fusion proteins in the context of phage display is known (see, e.g., U.S. Pat. No. 5,571,698). Similar techniques may be employed with the instant invention by linking the binding polypeptide of interest to an anchor for the periplasmic face of the cytoplasmic membrane instead of, for example, the amino-terminal domain of the gene III coat protein of the filamentous phage M13, or another surface-associated molecule. Such fusions can be mutated to form a library of structurally related fusion proteins that are expressed in low quantity on the periplasmic face of the cytoplasmic membrane in accordance with the invention. As such, techniques for the creation of heterogeneous collections of candidate molecules which are well known to those of skill in the art in conjunction with phage display, can be adapted for use with the invention. Those of skill in the art will recognize that such adaptations will include the use of bacterial elements for expression of fusion proteins anchored to the periplasmic face of the inner membrane, including, promoter, enhancers or leader sequences. The current invention provides the advantage relative to phage display of not requiring the use of phage or expression of molecules on the outer cell surface, which may be poorly expressed or may be deleterious to the host cell.

Examples of techniques that could be employed in conjunction with the invention for creation of diverse candidate binding proteins and/or antibodies include the techniques for expression of immunoglobulin heavy chain libraries described in U.S. Pat. No. 5,824,520. In this technique, a single chain antibody library is generated by creating highly divergent, synthetic hypervariable regions. Similar techniques for antibody display are given by U.S. Pat. No. 5,922,545. These sequences may then be fused to nucleic acids encoding an anchor sequence for the periplasmic face of the inner membrane of Gram negative bacteria for the expression of anchored fusion polypeptides.

Methods for creation of fusion proteins are well known to those of skill in the art (see, for example, U.S. Pat. No. 5,780,279). One means for doing so comprises constructing a gene fusion between a candidate binding polypeptide and an anchor sequence and mutating the binding protein encoding nucleic acid at one or more codons, thereby generating a family of mutants. The mutated fusion proteins can then be expressed in large populations of bacteria. Those bacteria in which a target ligand binds, can then be isolated and the corresponding nucleic acid encoding the binding protein can be cloned.

V. Automated Screening with Flow Cytometry

In one embodiment of the invention, fluorescence activated cell sorting (FACS) screening or other automated flow cytometric techniques may be used for the efficient isolation of a bacterial cell comprising a target ligand bound to a candidate molecule and linked to the outer face of the cytoplasmic membrane of the bacteria. Such a cell may have had its outer membrane removed prior to screening. Instruments for carrying out flow cytometry are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

Flow cytometric techniques in general involve the separation of cells or other particles in a liquid sample. Typically, the purpose of flow cytometry is to analyze the separated particles for one or more characteristics thereof, for example, presence of a target ligand or other molecule. The basis steps of flow cytometry involve the direction of a fluid sample through an apparatus such that a liquid stream passes through a sensing region. The particles should pass one at a time by the sensor and are categorized base on size, refraction, light scattering, opacity, roughness, shape, fluorescence, etc.

Rapid quantitative analysis of cells proves useful in biomedical research and medicine. Apparati permit quantitative multiparameter analysis of cellular properties at rates of several thousand cells per second. These instruments provide the ability to differentiate among cell types. Data are often displayed in one-dimensional (histogram) or two-dimensional (contour plot, scatter plot) frequency distributions of measured variables. The partitioning of multiparameter data files involves consecutive use of the interactive one- or two-dimensional graphics programs.

Quantitative analysis of multiparameter flow cytometric data for rapid cell detection consists of two stages: cell class characterization and sample processing. In general, the process of cell class characterization partitions the cell feature into cells of interest and not of interest. Then, in sample processing, each cell is classified in one of the two categories according to the region in which it falls. Analysis of the class of cells is very important, as high detection performance may be expected only if an appropriate characteristic of the cells is obtained.

Not only is cell analysis performed by flow cytometry, but so too is sorting of cells. In U.S. Pat. No. 3,826,364, an apparatus is disclosed which physically separates particles, such as functionally different cell types. In this machine, a laser provides illumination which is focused on the stream of particles by a suitable lens or lens system so that there is highly localized scatter from the particles therein. In addition, high intensity source illumination is directed onto the stream of particles for the excitation of fluorescent particles in the stream. Certain particles in the stream may be selectively charged and then separated by deflecting them into designated receptacles. A classic form of this separation is via fluorescent-tagged antibodies, which are used to mark one or more cell types for separation.

Other examples of methods for flow cytometry that could include, but are not limited to, those described in U.S. Pat. Nos. 4,284,412; 4,989,977; 4,498,766; 5,478,722; 4,857,451; 4,774,189; 4,767,206; 4,714,682; 5,160,974; and 4,661,913, each of the disclosures of which are specifically incorporated herein by reference.

For the present invention, a beneficial aspect of flow cytometry is that multiple rounds of screening can be carried out sequentially. Cells may be isolated from an initial round of sorting and immediately reintroduced into the flow cytometer and screened again to improve the stringency of the screen. Another advantage known to those of skill in the art is that nonviable cells can be recovered using flow cytometry. Since flow cytometry is essentially a particle sorting technology, the ability of a cell to grow or propagate is not necessary. Techniques for the recovery of nucleic acids from such non-viable cells are well known in the art and may include, for example, use of template-dependent amplification techniques including PCR.

VI. Nucleic Acid-Based Expression Systems

Nucleic acid-based expression systems may find use, in certain embodiments of the invention, for the expression of recombinant proteins. For example, one embodiment of the invention involves transformation of Gram negative bacteria with the coding sequences of fusion polypeptides comprising a candidate antibody or other binding protein having affinity for a selected ligand and the expression of such molecules on the cytoplasmic membrane of the Gram negative bacteria together with a target ligand expressed in the periplasm. In other embodiments of the invention, expression of such coding sequences may be carried, for example, in eukaryotic host cells for the preparation of isolated binding proteins having specificity for the target ligand. The isolated protein could then be used in one or more therapeutic or diagnostic applications.

A. Methods of Nucleic Acid Delivery

Certain aspects of the invention may comprise delivery of nucleic acids to target cells. For example, bacterial host cells may be transformed with nucleic acids encoding candidate molecules potentially capable binding a target ligand, In particular embodiments of the invention, it may be desired to target the expression to the cytoplasmic membrane of the bacteria. Transformation of eukaryotic host cells may similarly find use in the expression of various candidate molecules identified as capable of binding a target ligand.

Suitable methods for nucleic acid delivery for transformation of a cell are believed to include virtually any method by which a nucleic acid (e.g., DNA) can be introduced into such a cell, or even an organelle thereof. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

1. Electroporation

In certain embodiments of the present invention, a nucleic acid is introduced into a cell via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

2. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

B. Vectors

Vectors may find use with the current invention, for example, in the transformation of a Gram negative bacterium with a nucleic acid sequence encoding a candidate polypeptide which one wishes to screen for ability to bind a target ligand. In one embodiment of the invention, an entire heterogeneous “library” of nucleic acid sequences encoding target polypeptides may be introduced into a population of bacteria, thereby allowing screening of the entire library. The term “vector” is used to refer to a carrier nucleic acid molecule into which a nucleic acid sequence can be inserted for introduction into a cell where it can be replicated. A nucleic acid sequence can be “exogenous,” or “heterologous”, which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art may construct a vector through standard recombinant techniques, which are described in Maniatis et al., 1988 and Ausubel et al., 1994, both of which references are incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (see U.S. Pat. No. 4,683,202, U.S. Pat. No. 5,928,906, each incorporated herein by reference). Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. One example of such promoter that may be used with the invention is the E. coli arabinose promoter. Those of skill in the art of molecular biology generally are familiar with the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

3. Multiple Cloning Sites

Vectors can include a multiple cloning site (MCS), which is a nucleic acid region that contains multiple restriction enzyme sites, any of which can be used in conjunction with standard recombinant technology to digest the vector (see Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Termination Signals

The vectors or constructs prepared in accordance with the present invention will generally comprise at least one termination signal. A “termination signal” or “terminator” is comprised of the DNA sequences involved in specific termination of an RNA transcript by an RNA polymerase. Thus, in certain embodiments, a termination signal that ends the production of an RNA transcript is contemplated. A terminator may be necessary in vivo to achieve desirable message levels.

Terminators contemplated for use in the invention include any known terminator of transcription described herein or known to one of ordinary skill in the art, including but not limited to, for example, rhp dependent or rho independent terminators. In certain embodiments, the termination signal may be a lack of transcribable or translatable sequence, such as due to a sequence truncation.

5. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

6. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

C. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

In particular embodiments of the invention, a host cell is a Gram negative bacterial cell. These bacteria are suited for use with the invention in that they posses a periplasmic space between the inner and outer membrane and, particularly, the aforementioned inner membrane between the periplasm and cytoplasm, which is also known as the cytoplasmic membrane. As such, any other cell with such a periplasmic space could be used in accordance with the invention. Examples of Gram negative bacteria that may find use with the invention may include, but are not limited to, E. coli, Pseudomonas aeruginosa, Vibrio cholera, Salmonella typhimurium, Shigella flexneri, Haemophilus influenza, Bordotella pertussi, Erwinia amylovora, Rhizobium sp. The Gram negative bacterial cell may be still further defined as bacterial cell which has been transformed with the coding sequence of a fusion polypeptide comprising a candidate binding polypeptide capable of binding a selected ligand. The polypeptide is anchored to the outer face of the cytoplasmic membrane, facing the periplasmic space, and may comprise an antibody coding sequence or another sequence. One means for expression of the polypeptide is by attaching a leader sequence to the polypeptide capable of causing such directing.

Numerous prokaryotic cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for bacteriophage.

Many host cells from various cell types and organisms are available and would be known to one of skill in the art. Similarly, a viral vector may be used in conjunction with a prokaryotic host cell, particularly one that is permissive for replication or expression of the vector. Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

D. Expression Systems

Numerous expression systems exist that comprise at least a part or all of the compositions discussed above. Such systems could be used, for example, for the production of a polypeptide product identified in accordance with the invention as capable of binding a particular ligand. Prokaryote- -based systems can be employed for use with the present invention to produce nucleic acid sequences, or their cognate polypeptides, proteins and peptides. Many such systems are commercially and widely available. Other examples of expression systems comprise of vectors containing a strong prokaryotic promoter such as T7, Tac, Trc, BAD, lambda pL, Tetracycline or Lac promoters, the pET Expression System and an E. coli expression system.

E. Candidate Binding Proteins and Antibodies

In certain aspects of the invention, candidate antibodies or other recombinant polypeptides, including proteins and short peptides potentially capable of binding a target ligand are expressed on the cytoplasmic membrane of a host bacterial cell. By expression of a heterogeneous population of such antibodies or other binding polypeptides, those antibodies having a high affinity for a target ligand may be identified. The identified antibodies may then be used in various diagnostic or therapeutic applications, as described herein.

As used herein, the term “antibody” is intended to refer broadly to any immunologic binding agent such as IgG, IgM, IgA, IgD and IgE. The term “antibody” is also used to refer to any antibody-like molecule that has an antigen binding region, and includes antibody fragments such as Fab′, Fab, F(ab′)₂, single domain antibodies (DABs), Fv, scFv (single chain Fv), and engineering multivalent antibody fragments such as dibodies, tribodies and multibodies. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Once an antibody having affinity for a target ligand is identified, the antibody or ligand binding polypeptide may be purified, if desired, using filtration, centrifugation and various chromatographic methods such as HPLC or affinity chromatography. Fragments of such polypeptides, including antibodies, can be obtained from the antibodies so produced by methods which include digestion with enzymes, such as pepsin or papain, and/or by cleavage of disulfide bonds by chemical reduction. Alternatively, antibody or other polypeptides, including protein fragments, encompassed by the present invention can be synthesized using an automated peptide synthesizer.

A molecular cloning approach comprises one suitable method for the generation of a heterogeneous population of candidate antibodies that may then be screened in accordance with the invention for affinity to target ligands. In one embodiment of the invention, combinatorial immunoglobulin phagemid can be prepared from RNA isolated from the spleen of an animal. By immunizing an animal with the ligand to be screened, the assay may be targeted to the particular antigen. The advantages of this approach over conventional techniques are that approximately 10⁴ times as many antibodies can be produced and screened in a single round, and that new specificities are generated by H and L chain combination which further increases the chance of finding appropriate antibodies.

VII. Manipulation and Detection of Nucleic Acids

In certain embodiments of the invention, it may be desired to employ one or more techniques for the manipulation, isolation and/or detection of nucleic acids. Such techniques may include, for example, the preparation of vectors for transformation of host cells as well as methods for cloning selected nucleic acid segments from a transgenic cell. Methodology for carrying out such manipulations will be well known to those of skill in the art in light of the instant disclosure.

Nucleic acids used as a template for amplification may be isolated from cells, tissues or other samples according to standard methodologies (Sambrook et al., 1989). In certain embodiments, analysis may be performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to a selected nucleic acid sequence are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., 1988, each of which is incorporated herein by reference in their entirety.

A reverse transcriptase PCR amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application 320 308, incorporated herein by reference in its entirety. U.S. Pat. No. 4,883,750 describes a method similar to LCR for binding probe pairs to a target sequence. A method based on PCR and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) discloses a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Demonstration of Anchored Periplasmic Expression to Target Small Molecules and Peptides

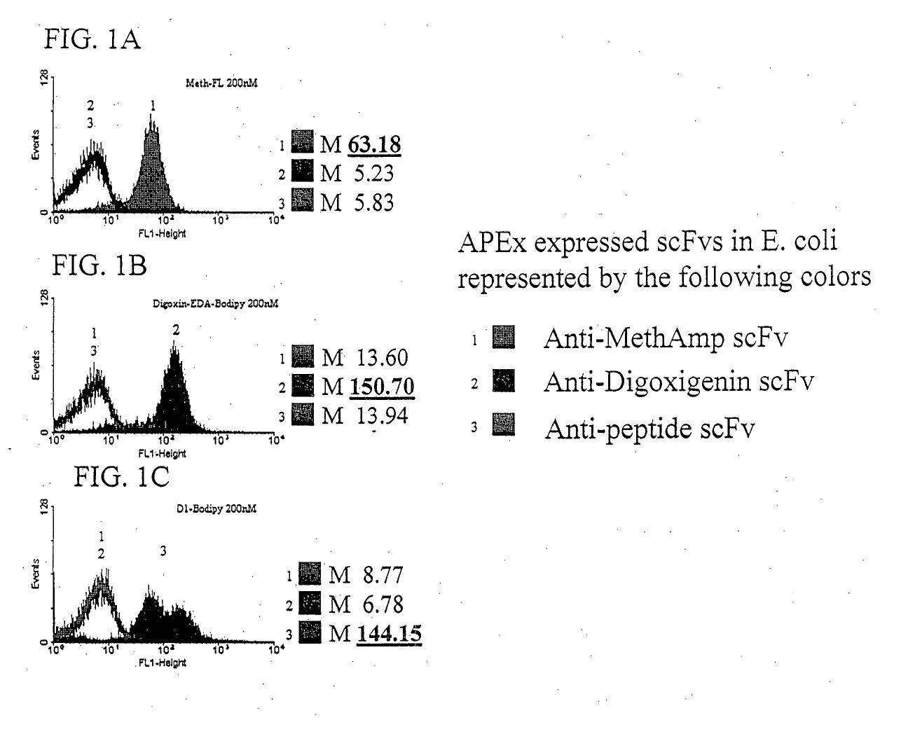

The ability of scFvs displayed by APEx to target small molecules and peptides is shown in FIGS. 1A-1B and in FIG. 1C, respectively. Three cultures of Escherichia coli containing fusions of the first six amino acids of NlpA (to serve as a inner membrane targeting sequence for APEx analysis) to either an anti-methamphetamine, anti-digoxin, or anti-peptide scfv were grown up and induced for protein expression as described below. Cells of each construct were then labeled in 5×PBS buffer with 200 nM concentrations of methamphetamine-FL (FIG. 1A), digoxigenin-bodipy (FIG. 1B), or 200 nM peptide (18mer)-BodipyFL (FIG. 1C). The data presented shows a histogram representation of 10,000 events from each of the labeled cell cultures. The results demonstrate the ability of scfvs displayed by APEx to bind to their specific antigen conjugated fluorophore, with minimal crossreactivity to non-specific ligands.

### Example 2

### Demonstration of Recognition of Ab Fragments by Anchored Periplasmic Expression

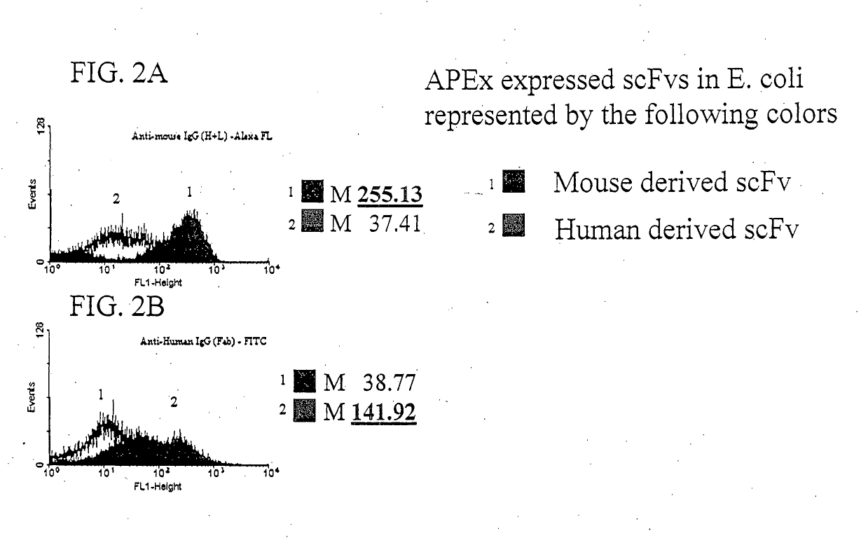

To demonstrate that the scFv is accessible to larger proteins, it was first demonstrated that polyclonal antibody serum against human Ab fragments or mouse Ab fragments would recognize scFvs derived from each displayed on the E. coli inner membrane by anchored periplasmic expression. Escherichia coli expressing a mouse derived scFv via anchored periplasmic expression (FIG. 2A) or expressing a human derived scFv via anchored periplasmic expression (FIG. 2B) were labeled as described below with either anti-mouse polyclonal IgG (H+L)-Alexa-FL or anti-human polyclonal IgG (Fab)-FITC. Results (FIG. 2A, 2B) in the form of histogram representations of 10000 events of each demonstrated that the anti-human polyclonal (approximately 150 kDa in size) recognized the human derived scFv specifically while the anti-mouse polyclonal (150 kDa) recognized the mouse derived scFv.

### Example 3

### Demonstration of the Ability of scFvs Displayed by Anchored Periplasmic Expression to Specifically Bind Large Antigen Conjugated Fluorophores

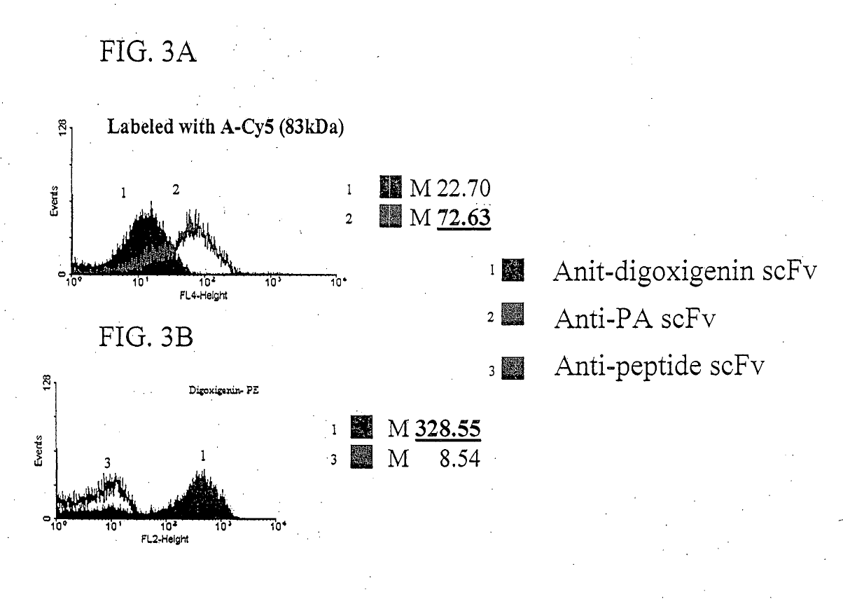

To demonstrate the ability of scFvs displayed via anchored periplasmic expression to specifically bind to large antigen conjugated fluorophores, E. coli were induced and labeled as described below expressing, via anchored periplasmic expression, an anti-protective antigen (PA) scFv (PA is one component of the anthrax toxin: a 83 kDa protein) or an anti-digoxigenin scFv. Histogram data of 10,000 events demonstrated specific binding to a PA-Cy5 antigen conjugated fluorophore as compared to the cells expressing the an anti-digoxigenin scFv (FIG. 3A). To further illustrate this point, digoxigenin was coupled to phycoerythrin (PE), a 240 kDa fluorescent protein. Cells were labeled with this conjugate as described below. It was found that E. coli (10,000 events) expressing the anti-digoxigenin scFv via anchored periplasmic expression were labeled with the large PE-digoxigenin conjugate while those expressing a non-specific scFv via anchored periplasmic expression show little fluorescence (FIG. 3B).

### Example 4

### Demonstration of Selecting for Improved scFv Variants from a Library of scFvs by Flow Cytometric Selection

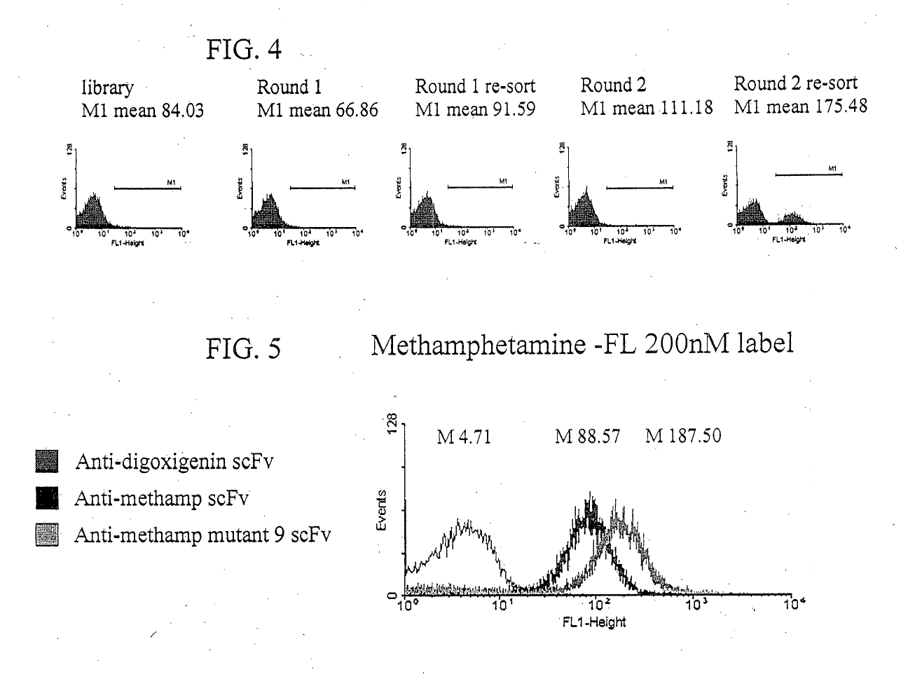

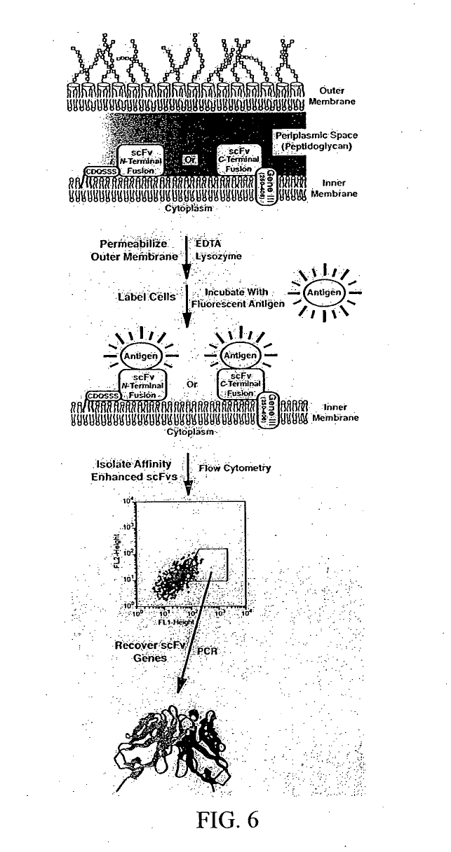

Scans were carried out of polyclonal Escherichia coli expressing, via anchored periplasmic expression, a mutagenic library of an scFv with affinity to methamphetamine. Through two rounds of sorting and re-sorting using a Methamphetamine conjugated fluorophore, a sub-population of the library was isolated. (FIG. 4) Individual clones from this library were labeled with the same Methamphetamine fluorophore and analyzed as described below. Shown in FIG. 5 is an example of a clone, designated mutant 9, that had a higher mean FL signal than the parent anti-methamphetamine scFv.

### Example 5

### Materials and Methods

A. Vector Construction

The leader peptide and first six amino acids of the mature NlpA protein were generated by whole cell PCR (Perken Elmer) on XL1-blue Escherichia coli, (Stratagene) using primers

| | |
|---|---|
| BRH#08 | |
| 5′ GAAGGAGATATACATATGAAACTGACAACACATC | (SEQ ID NO:6) |
| ATCTA 3′ | |
| and | |
| BRH#9 | |
| 5′ CTGGGCCATGGCCGGCTGGGCCTCGCTGCTACTC | (SEQ ID NO:7) |
| TGGTCGCAACC 3′, | |

VENT polymerase (New England Biolabs) and dNTPs (Roche). This was then cut with Nde1 and Sfi1 restriction endonucleases and cloned between a lac promoter and a multiple cloning site (MCS) in a E. coli expression vector with the following elements down stream of the MCS: myc and his tag, Cm resistance marker, colE1 origin and lac I. ScFvs of interest were then cloned into the MCS and the vector was transformed into AbleC E. coli (Stratagene).

B. Expression

E. coli cells are inoculated in TB media+2% glucose and 30 mg/l chloramphenicol to an OD600 of 0.1. Cells are grown for 2 hours at 37 C and then cooled to 25 C for 30 minutes. They are then induced at 25 C with 1 mM IPTG for 4 hrs.

Mutagenic libraries of scFv sequences were constructed using mutagenic PCR methods as described by Fromant M, et al. (1995) utilizing the original scFv sequence as a template. These mutagenic products were then cloned into the above mentioned APEx expression vector, transformed into ABLEC E. coli and plated on agar plates with SOC media containing 2% glucose and 30 ug/ml chloramphenicol. Following overnight incubation at 30 C, the E. coli were scraped from the plates, frozen in 15% glycerol aliquots and stored at −80 C for future flow cytometric sorting.

C. Labeling Strategies

Following induction, cells are either incubated in 5×PBS with 200 nM probe for 45 minutes or are resuspended in 350 μl of 0.75M sucrose, 100 mM Tris. 35 μl of lysozyme at 10 mg/ml is then added followed by 700 μl of 1 mM EDTA added dropwise with gentle shaking. This is allowed to sit on ice for 10 min followed by the addition of 50 μl of 0.5M MgCl₂. After an additional 10 minutes on ice the suspension is centrifuged at 13,200 g for 1 minute, decanted and resuspended in 500 μl 1×PBS. The cells are then labeled with 200 nM of probe for 45 minutes, and are then analyzed by flow cytometry and selected for improved fluorescence.

D. Strains and Plasmids

Strain ABLE™C (Stratagene) was used for screening with APEx. E. coli strains TG1 and HB2151 were provided with the Griffin library. ABLE™C and ABLE™K were purchased from Stratagene and helper phage M13K07 from Pharmacia. A positive control for FACS analysis of a phage display vehicle was constructed by replacing a pre-existing scFv in pHEN2 with the 26.10 scFv to create pHEN2.dig. The negative control was pHEN2.thy bearing the anti-thyroglobulin scFv provided with the Griffin.1 library. The P_{tac} vector was a derivative of pIMS120 (Hayhurst, 2000).

E. Phage Panning

The Griffin.1 library is a semi-synthetic scFv library derived from a large repertoire of human heavy and light chains with part or all of the CDR3 loops randomly mutated and recombined in vivo (Griffiths et al., 1994). The library represents one potential source of candidate binding polypeptides for screening by anchored periplasmic expression in accordance with the invention. The library was rescued and subjected to five rounds of panning according to the web-site instruction manual (www.mrc-cpe.cam.ac.uk/˜phage/glp.html), summarized in Example 9, below. Immunotubes were coated with 10 μgml⁻¹ digoxin-BSA conjugate and the neutralized eluates were halved and used to infect either TG-1 for the next round of phage panning, or ABLE™ C for FACS analysis.

Eluate titers were monitored to indicate enrichment of antigen binding phage. To confirm reactivity, a polyclonal phage ELISA of purified, titer normalized phage stocks arising from each round was performed on digoxin-ovalbumin conjugate. The percentage of positive clones arising in rounds 3, 4 and 5 was established by monoclonal phage ELISA of 96 isolates after each round. A positive was arbitrarily defined as an absorbance greater than 0.5 with a background signal rarely above 0.01. MvaI fingerprinting was applied to 24 positive clones from rounds 3, 4 and 5.

F. FACS Screening

For scanning with APEx expression, glycerol stocks of E. coli carrying the APEx construct were grown and labeled as described in section B and C. Following labeling cells were washed once in PBS and scanned. In the aforementioned studies using bodipy or FL labeled antigen, a 488 nm laser for excitation was used, while with Cy5 a 633 nm laser was used. Scanning was accomplished on a FACSCalibur (BD) using the following instrument settings: Sidescatter trigger V 400, Threshold 250, Forward scatter E01, FL1 V 400 FL2 V 400 (488 nm ex), FL4 V 700 (633 nm ex).

Sorting with APEx expression was as follows: all sorts were performed using a MoFlo FC (Cytomation). Previously described libraries were grown and labeled as described in section B and C, washed once with PBS and sorted for increased FL intensity. Subsequent rounds of sorting were applied until polyclonal scans of the population demonstrate enrichment. (See FIG. 4) Individual clones were then picked and analyzed for FL activity.

For other studies, an aliquot of phagemid containing, ABLE™C glycerol stock was scraped into 1 ml of 2×TY (2% glucose, 100 μgml⁻¹ ampicillin) to give an OD at 600 nm of approximately 0.1 cm⁻¹. After shaking vigorously at 37° C. for 2 h, IPTG was added to 1 mM and the culture shaken at 25° C. for 4 h. 50 μl of culture was labeled with 100 nM BODIPY™-digoxigenin (Daugherty et al., 1999) in 1 ml of 5×PBS for 1 h at room temperature with moderate agitation. For the last 10 min of labeling, propidium iodide was added to 2 μg/ml⁻¹. Cells were pelleted and resuspended in 100 μl of labeling mix. Scanning was performed with Becton-Dickinson FACSort, collecting 10⁴ events at 1500 s⁻¹.

For FACS library sorting, the cells were grown in terrific broth and induced with 0.1 mMIPTG. Sorting was performed on 10⁶ events (10⁷ for round 2) in exclusion mode at 1000 s⁻¹. Collected sort liquor was passed through 0.7 μm membrane filters and colonies allowed to grow after placing the filter on top of SOC agar plus appropriate antibiotics at 30° C. for 24 h.

G. Analysis of Phage Clones

Screening phage particles by ELISA is summarized as follows. Binding of phage in ELISA is detected by primary sheep anti-M13 antisera (CP laboratories or 5 prime-3 prime) followed by a horseradish peroxidase (HRP) conjugated anti-sheep antibody (Sigma). Alternatively, a HRP-anti-M13 conjugate can be used (Pharmacia). Plates can be blocked with 2% MPBS or 3% BSA-PBS. For the polyclonal phage ELISA, the technique is generally as follows: coat MicroTest III flexible assay plates (Falcon) with 100 μl per well of protein antigen. Antigen is normally coated overnight at 4° C. at a concentration of 10-100 μg/ml in either PBS or 50 mM sodium hydrogen carbonate, pH 9.6. Rinse wells 3 times with PBS, by flipping over the ELISA plates to discard excess liquid, and fill well with 2% MPBS or 3% BSA-PBS for 2 hr at 37° C. Rinse wells 3 times with PBS. Add 10 μl PEG precipitated phage from the stored aliquot of phage from the end of each round of selection (about 10¹⁰ tfu.). Make up to 100 μl with 2% MPBS or 3% BSA-PBS. Incubate for 90 min at rt. Discard the test solution and wash three times with PBS-0.05% Tween 20, then 3 times with PBS. Add appropriate dilution of HRP-anti-M13 or sheep anti-M13 antisera in 2% MPBS or 3% BSA-PBS. Incubate for 90 min at rt, and wash three times with PBS-0.05% Tween 20, then 3 times with PBS. If sheep anti-M13 antisera is used, incubate for 90 min at rt, with a suitable dilution of HRP-anti-sheep antisera in 2% MPBS or 3% BSA and wash three times with PBS-0.05% Tween 20, then 3 times with PBS. Develop with substrate solution (100 μg/ml TMB in 100 mM sodium acetate, pH 6.0, add 10 μl of 30% hydrogen peroxide per 50 ml of this solution directly before use). Add 100 μl to each well and leave at rt for 10 min. A blue color should develop. Stop the reaction by adding 50 μl 1 M sulfuric acid. The color should turn yellow. Read the OD at 450 nm and at 405 nm. Subtract OD 405 from OD 450.

Monoclonal phage ELISA can be summarized as follows. To identify monoclonal phage antibodies the pHEN phage particles need to be rescued: Inoculate individual colonies from the plates in C10 (after each round of selection) into 100 μl 2×TY containing 100 μg/ml ampicillin and 1% glucose in 96-well plates (Corning ‘Cell Wells’) and grow with shaking (300 rpm.) overnight at 30° C. Use a 96-well transfer device to transfer a small inoculum (about 2 μl) from this plate to a second 96-well plate containing 200 μl of 2×TY containing 100 μg/ml ampicillin and 1% glucose per well. Grow shaking at 37° C. for 1 hr. Make glycerol stocks of the original 96-well plate, by adding glycerol to a final concentration of 15%, and then storing the plates at −70° C. To each well (of the second plate) add VCS-M13 or M13KO7 helper phage to an moi of 10. Stand for 30 min at 37° C. Centrifuge at 1,800 g for 10 min, then aspirate off the supernatant. Resuspend pellet in 200 μl 2×TY containing 100 μg/ml ampicillin and 50 μg/ml kanamycin. Grow shaking overnight at 30° C. Spin at 1,800 g for 10 min and use 100 μl of the supernatant in phage ELISA as detailed above.

Production of antibody fragments is summarized as follows: the selected pHEN needs to be infected into HB2151 and then induced to give soluble expression of antibody fragments for ELISA. From each selection take 10 μl of eluted phage (about 10⁵ t.u.) and infect 200 μl exponentially growing HB2151 bacteria for 30 min at 37° C. (waterbath). Plate 1, 10, 100 μl, and 1:10 dilution on TYE containing 100 μg/ml ampicillin and 1% glucose. Incubate these plates overnight at 37° C. Pick individual colonies into 100 μl 2×TY containing 100 μg/ml ampicillin and 1% glucose in 96-well plates (Corning ‘Cell Wells’), and grow with shaking (300 rpm.) overnight at 37° C. A glycerol stock can be made of this plate, once it has been used to inoculate another plate, by adding glycerol to a final concentration of 15% and storing at −70° C. Use a 96-well transfer device to transfer a small inocula (about 2 μl) from this plate to a second 96-well plate containing 200 μl fresh 2×TY containing 100 μg/ml ampicillin and 0.1% glucose per well. Grow at 37° C., shaking until the OD at 600 nm is approximately 0.9 (about 3 hr). Once the required OD is reached add 25 μl 2×TY containing 100 μg/ml ampicillin and 9 mM IPTG (final concentration 1 mM IPTG). Continue shaking at 30° C. for a further 16 to 24 hr. Coat MicroTest III flexible assay plates (Falcon) with 100 μl per well of protein antigen.

Antigen is normally coated overnight at rt at a concentration of 10-100 μg/ml in either PBS or 50 mM sodium hydrogen carbonate, pH 9.6. The next day rinse wells 3 times with PBS, by flipping over the ELISA plates to discard excess liquid, and block with 200 μl per well of 3% BSA-PBS for 2 hr at 37° C. Spin the bacterial plate at 1,800 g for 10 min and add 100 μl of the supernatant (containing the soluble scFv) to the ELISA plate for 1 hr at rt. Discard the test solution and wash three times with PBS. Add 50 μl purified 9E10 antibody (which detects myc-tagged antibody fragments) at a concentration of 4 μg/ml in 1% BSA-PBS and 50 μl of a 1:500 dilution of HRP-anti-mouse antibody in 1% BSA-PBS. Incubate for 60 min at rt, and wash three times with PBS-0.05% Tween 20, then 3 times with PBS. Develop with substrate solution (100 μg/ml TMB in 100 mM sodium acetate, pH 6.0. Add 10 μl of 30% hydrogen peroxide per 50 ml of this solution directly before use). Add 100 μl to each well and leave at rt for 10 min. A blue color should develop. Stop the reaction by adding 50 μl 1 M sulphuric acid. The color should turn yellow. Read the OD at 450 nm and at 405 nm. Subtract OD 405 from OD 450.

Inserts in the library can be screened by PCR screening using the primers designated LMB3: CAG GAA ACA GCT ATG AC (SEQ ID NO:1) and Fd seq1: GAA TTT TCT GTA TGA GG (SEQ ID NO:2). For sequencing of the VH and VL, use is recommend of the primers FOR_LinkSeq: GCC ACC TCC GCC TGA ACC (SEQ ID NO:3) and pHEN-SEQ: CTA TGC GGC CCC ATT CA (SEQ ID NO:4).

### Example 6

### Use of Anchored Periplasmic Expression to Isolate Antibodies with Over a 120-Fold Improvement in Affinity for the Bacillus anthracis Protective Antigen

The screening of large libraries requires a physical link between a gene, the protein it encodes, and the desired function. Such a link can be established using a variety of in vivo display technologies that have proven invaluable for mechanistic studies, for biotechnological purposes and for proteomics research (Hoess, 2001; Hayhurst and Georgiou, 2001; Wittrup, 2000).

APEx is an alternative approach that allows screening by flow cytometry (FC). FC combines high throughput with real-time, quantitative, multi-parameter analysis of each library member. With sorting rates on the order of more than 400 million cells per hour, commercial FC machines can be employed to screen libraries of the size accessible within the constraints of microbial transformation efficiencies. Furthermore, multi-parameter FC can provide valuable information regarding the function of each and every clone in the library in real time, thus helping to guide the library construction process and optimize sorting conditions (Boder and Wittrup, 2000; Daugherty et al., 2000).

Bacterial and yeast protein display in combination with FC has been employed for the engineering of high affinity antibodies to a variety of ligands (Daugherty et al., 1999; Boder et al., 2000). However, the requirement for the display of proteins on cell surfaces imposes a number of biological constraints that can impact library screening applications. Processes such as the unfolded protein response in eucaryotes or the stringency of protein sorting to the outer membrane of Gram-negative bacteria limit the diversity of the polypeptides that are actually compatible with surface display (Sagt et al., 2002; Sathopoulos et al., 1996). In addition, microbial surfaces are chemically complex structures whose macromolecular composition can interfere with protein:ligand recognition. This problem is particularly manifest in Gram-negative bacteria because the presence of lipopolysaccharides on the outer membrane presents a steric barrier to protein:ligand recognition, a fact that likely contributed to the evolution of specialized appendages, such as pili or fimbriae (Hultgren et al., 1996).

APEx overcomes the biological constraints and antigen access limitations of previous display strategies, enabling the efficient isolation of antibodies to virtually any size antigen. In APEx, proteins are tethered to the external (periplasmic) side of the E. coli cytoplasmic membrane as either N- or C-terminal fusions, thus eliminating biological constraints associated with the display of proteins on the cell surface. Following chemical/enzymatic permeabilization of the bacterial outer membrane, E. coli cells expressing anchored scFv antibodies can be specifically labeled with fluorescent antigens, of at least 240 kDa, and analyzed by FC. By using APEx the inventors have demonstrated the efficient isolation of antibodies with markedly improved ligand affinities, including an antibody fragment to the protective antigen of Bacillus anthracis with an affinity that was increased over 120-fold.

A. Anchored Periplasmic Expression and Detection of Ligand Binding

For screening applications, an ideal expression system should minimize cell toxicity or growth abnormalities that can arise from the synthesis of heterologous polypeptides (Daugherty et al., 2000). Use of APEx avoids the complications that are associated with transmembrane protein fusions (Miroux and Walker, 1996; Mingarro et al., 1997). Unlike membrane proteins, bacterial lipoproteins are not known to require the SRP or YidC pathways for membrane anchoring (Samuelson et al., 2000). Lipoproteins are secreted across the membrane via the Sec pathway and once in the periplasm, a diacylglyceride group is attached through a thioether bond to a cysteine residue on the C-terminal side of the signal sequence. The signal peptide is then cleaved by signal peptidase II, the protein is fatty acylated at the modified cysteine residue, and finally the lipophilic fatty acid inserts into the membrane, thereby anchoring the protein (Pugsley, 1993; Seydel et al., 1999; Yajushi et al., 2000).

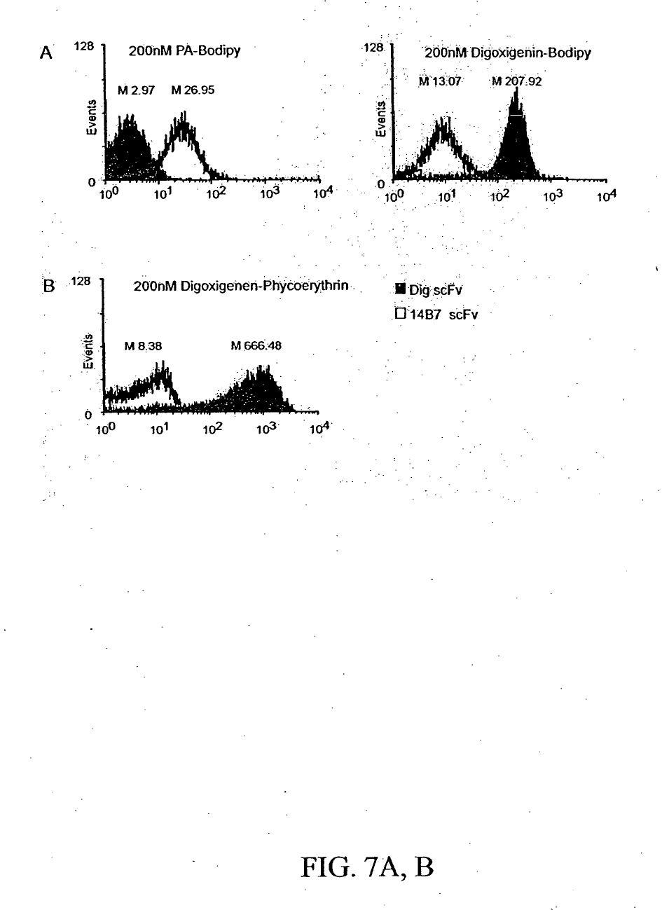

A sequence encoding the leader peptide and first six amino acids of the mature NlpA (containing the putative fatty acylation and inner membrane targeting sites) was employed for anchoring scFv antibodies to the periplasmic face of the inner membrane. NlpA is a non-essential E. coli lipoprotein that exclusively localizes to the inner membrane (Yu et al., 1986; Yamaguchi et al., 1988). Of particular note is the aspartate residue adjacent to the fatty acylated cysteine residue that is thought to be a consensus residue for inner membrane targeting (Yamaguchi et al., 1988). NlpA fusions to the 26-10 anti-digoxin/digoxigenin (Dig) scFv and to the anti-B. anthracis protective antigen (PA) 14B7 scFv were constructed and expressed from a lac promoter in E. coli. Following induction of the NlpA-[scFv] synthesis using IPTG, the cells were incubated with EDTA and lysozyme to disrupt the outer membrane and the cell wall. The permeabilized cells were mixed with the respective antigens conjugated to the fluorescent dye BODIPY™ (200 nM) and the cell fluorescence was determined by flow cytometry. Treated cells expressing the NlPA-[14B7 scFv] and the NlpA-[Dig scFv] exhibited an approximate 9-fold and 16-fold higher mean fluorescence intensity, respectively, compared to controls (FIG. 7A). Only background fluorescence was detected when the cells were mixed with unrelated fluorescent antigen, indicating negligible background binding under the conditions of the study.

To further evaluate the ability of antibody fragments anchored on the cytoplasmic membrane to bind bulky antigens, the inventors examined the ability of the NlpA-[Dig scFv] to recognize digoxigenin conjugated to the 240 kDa fluorescent protein phycoerythrin (PE). The conjugate was mixed with cells expressing NlpA-[Dig scFv] and treated with EDTA-lysozyme. A high cell fluorescence was observed indicating binding of digoxigenin-PE conjugate by the membrane anchored antibody (FIG. 7B). Overall, the accumulated data demonstrated that in cells treated with Tris-EDTA-lysozyme, scFvs anchored on the cytoplasmic membrane can readily bind to ligands ranging from small molecules to proteins of at least up to 240 kDa in molecular weight. Importantly, labeling with digoxigenin-PE followed by one round of flow cytometry resulted in an over 500-fold enrichment of bacteria expressing NlpA-[Dig scFv] from cells expressing a similar fusion with a scFv having unrelated antigen specificity.

B. Library Screening by APEx

A library of 1×10⁷ members was constructed by error-prone PCR of the gene for the anti-PA 14B7 scFv and was fused to the NlpA membrane anchoring sequence. DNA sequencing of 12 library clones selected at random revealed an average of 2% nucleotide substitutions per gene. Following induction of NlpA-[14B7 mutant scFv] synthesis with IPTG, the cells were treated with Tris-EDTA-lysozyme, washed, and labeled with 200 nM PA-BODIPY™. Inner membrane integrity was monitored by staining with propidium iodide (PI). A total of 2×10⁸ bacteria were sorted using an ultra-high throughput Cytomation Inc. MoFlo droplet deflection flow cytometer selectively gating for low PI fluorescence (630 nm emission) and high BODIPY™ fluorescence. Approximately 5% of the cells sorted with the highest 530 nm fluorescence (FL1) were collected, immediately restained with PI alone and resorted as above. Since no antigen was added during this second sorting cycle, only cells expressing antibodies that have slow dissociation kinetics remain fluorescent. The plating efficiency of this population was low, presumably due to a combination of potential scFv toxicity (Somerville et al., 1994; Hayhurst and Harris, 1999), Tris-EDTA-lysozyme treatment and exposure to the high shear flow cytometry environment. Therefore, to avoid loss of potentially high affinity clones, DNA encoding scFvs was rescued by PCR amplification of the approximately 1×10⁴ fluorescent events recovered by sorting. It should be noted that the conditions used for PCR amplification result in the quantitative release of cellular DNA from the cells which have partially hydrolyzed cell walls due to the Tris-EDTA-lysozyme treatment during labeling. Following 30 rounds of PCR amplification, the DNA was ligated into pAPEx1 and transformed into fresh E. coli. A second round of sorting was performed exactly as above, except that in this case only the most fluorescent 2% of the population was collected and then immediately resorted to yield approximately 5,000 fluorescent events.

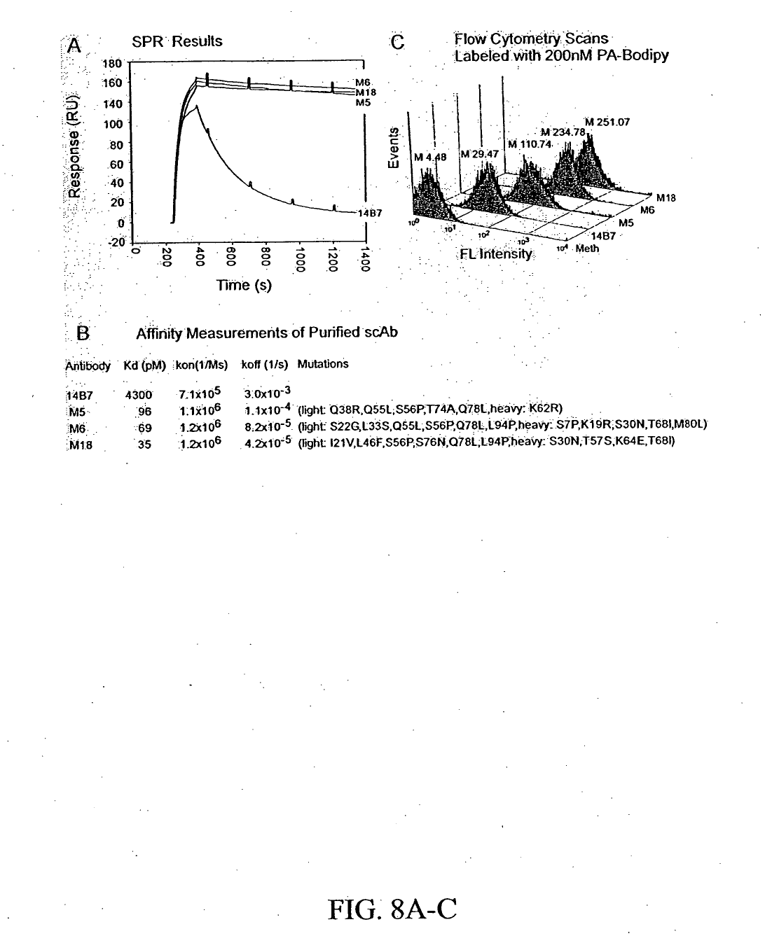

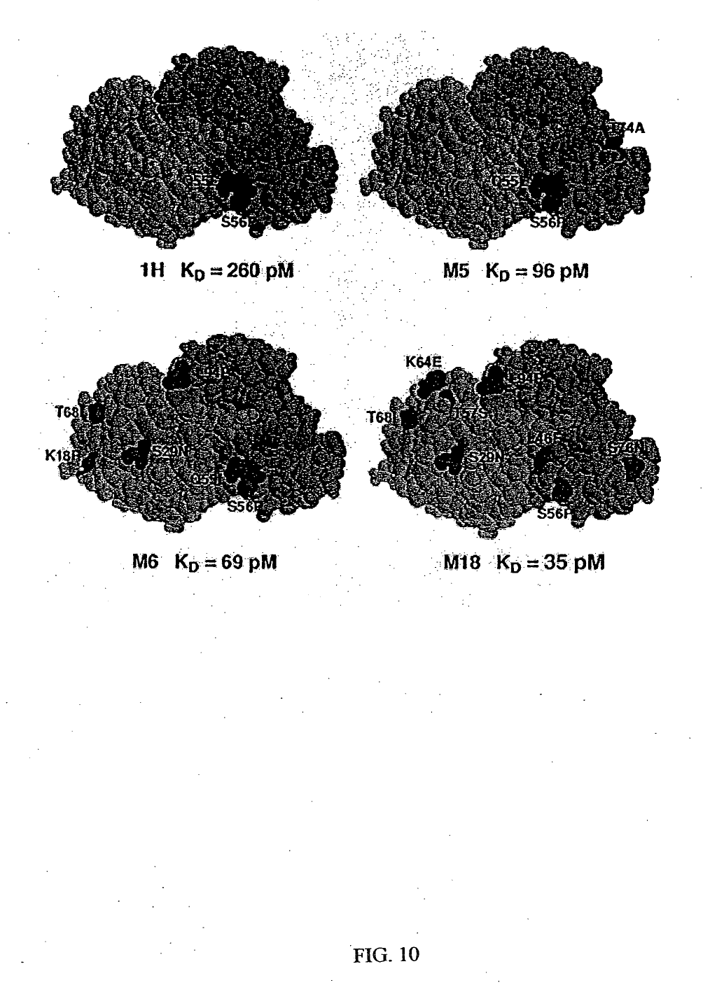

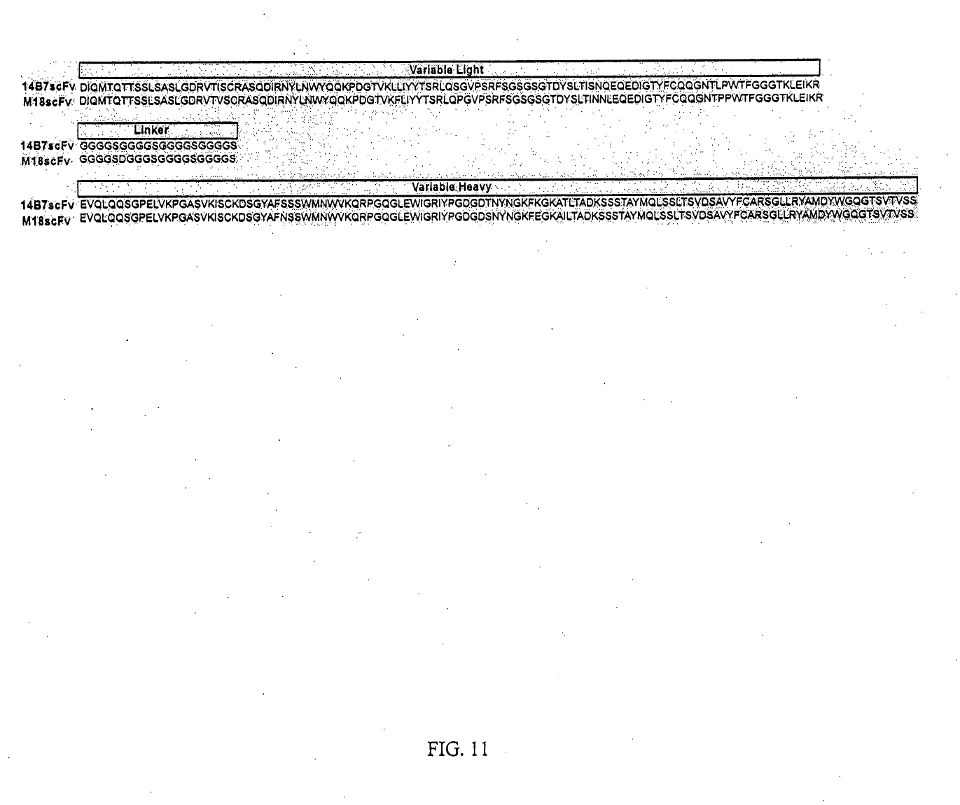

The scFv DNA from the second round was amplified by PCR and ligated into pMoPac16 (Hayhurst et al., 2003) for expression of the antibody fragments in soluble form in the scAb format. A scAb antibody fragment is comprised of an scFv in which the light chain is fused to a human kappa constant region. This antibody fragment format exhibits better periplasmic solubility compared to scFvs (Maynard et al., 2002; Hayhurst, 2000). 20 clones in the scAb format were picked at random and grown in liquid cultures. Following induction with IPTG, periplasmic proteins were isolated and the scAb proteins were rank-ordered with respect to their relative antigen dissociation kinetics, using surface plasmon resonance (SPR) analysis. 11 of the 20 clones exhibited slower antigen dissociation kinetics compared to the 14B7 parental antibody. The 3 scAbs with the slowest antigen dissociation kinetics were produced in large scale and purified by Ni chromatography followed by gel filtration FPLC. Interestingly, all the library-selected clones exhibited excellent expression characteristics and resulted in yields of between 4-8 mg of purified protein per L in shake flask culture. Detailed BIACore analysis indicated that all 3 clones exhibit a substantially lower K_{D} for PA compared to the parental 14B7 antibody (FIGS. 8A and 8B). The improved K_{D} resulted primarily from slower antigen dissociation, (i.e. slower k_{off}). The highest affinity clone, M18, exhibited K_{D} of 35 pM, with a k_{off} of 4.2×10⁻⁵ M⁻¹ sec⁻¹ which corresponds to a M18-PA half life of 6.6 hours. This represents over 120-fold affinity improvement compared to the parental antibody 14B7 (K_{D}=4.3 nM as determined by BIACore 3000). The mutations identified are given in FIG. 8B and a schematic showing the conformation of the 1H, M5, M6 and M18 antibodies is given in FIG. 10. The mutations for M5 were as follows: in the light chain, Q38R, Q55L, S56P, T74A, Q78L and in the heavy chain, K62R. For M6, the mutations were as follows: S22G, L33S, Q55L, S56P, Q78L AND L94 P, and in the heavy chain, S7P, K19R, S30N, T68I and M80L. For M18, the mutations were as follows: in the light chain, 121V, L46F, S56P, S76N, Q78L and L94P, and in the heavy chain, S30N, T57S, K64E and T68. FIG. 11 shows an alignment of 14B7 scFv (SEQ ID NO:21) and M18 scFv (SEQ ID NO:23) sequences indicating the variable heavy and variable light chains and mutations made. The nucleic acids encoding these sequences are given in SEQ ID NO:20 and SEQ ID NO:22, respectively.

The fluorescence intensity of Tris-EDTA-lysozyme permeabilized cells expressing NlpA fusions to the mutant antibodies varied in proportion to the antigen binding affinity. (FIG. 8C) For example, cells expressing the NlpA-[M18 scFv] protein displayed a mean fluorescence of 250 whereas the cells that expressed the parental 14B7 scFv exhibited a mean fluorescence of 30, compared to a background fluorescence of around 5 (FIG. 8B). Antibodies with intermediate affinities displayed intermediate fluorescence intensities in line with their relative affinity rank. The ability to resolve cells expressing antibodies exhibiting dissociation constants as low as 35 pM provides a reasonable explanation for why three unique very high affinity variants could be isolated and is indicative of the fine resolution that can be obtained with flow cytometric analysis.

The 3 clones analyzed in detail, M5, M6 and M18, contained 7, 12, and 11 amino acid substitutions, respectively. In earlier studies using phage display (Maynard et al., 2002), the inventors isolated a variant of the 14B7 scFv by three cycles, each consisting of 1) mutagenic error prone PCR, 2) five rounds of phage panning and 3) DNA shuffling of the post-panning clones. The best clone isolated in that study, 1H, contained Q55L and S56P substitutions and exhibited a K_{D} of 150 pM (as determined by a BIACore3000). These two mutations likely increase the hydrophobicity of the binding pocket adding to the mounting evidence that an increase in hydrophobic interactions is a dominant effect in antibody affinity maturation (Li et al., 2003). The same amino acid substitutions are also found in the M5 and M6 clones isolated by APEx. However, the presence of the additional mutations in these two clones conferred a further increase in affinity. It is noteworthy that the M5, M6 and M18 were isolated following a single round of asexual PCR yet they all had higher affinity relative to the best antibody that could be isolated by phage display, even following multiple rounds of sexual mutagenesis and selection.

M18, the highest affinity clone isolated by APEx, contained the S56P mutation but lacked the Q55L substitution found in 1H, M5, and M6. When the Q55L substitution was introduced into M18 by site specific mutagenesis, the resultant ScAb exhibited a further improvement in antigen binding (K_{D}=21 pM) with a kₒₙ of 1.1×10⁶ M⁻¹ sec⁻¹ and a k_{off} of 2.4×10⁻⁵ sec⁻¹, corresponding to a complex half life of 11.6 hours. However, the introduction of this mutation reduced the yield of purified protein more than 5-fold to 1.2 mg/L in shake flask culture. The modified M18 sequence is given in SEQ ID NO:25 and the nucleic acid encoding this sequence is given in SEQ ID NO:24.

C. APEx of Phage Displayed scFv Antibodies

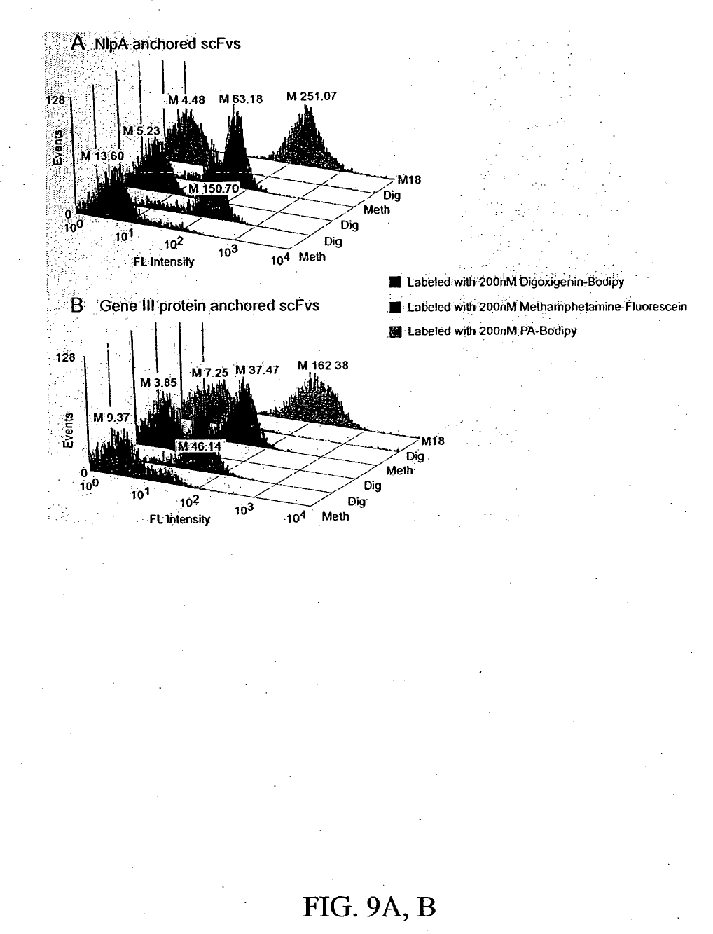

Numerous antibody fragments to important therapeutic and diagnostic targets have been isolated from repertoire libraries screened by phage display. It is desirable to develop a means for rapid antigen binding analysis and affinity maturation of such antibodies without the need for time consuming subcloning steps. Antibodies are most commonly displayed on filamentous phage via fusion to the N-terminus of the phage gene 3 minor coat protein (g3p) (Barbas et al., 1991). During phage morphogenesis, g3p becomes transiently attached to the inner membrane via its extreme C-terminus, before it can be incorporated onto the growing virion (Boeke and Model, 1982). The antibody fragments are thus both anchored and displayed in the periplasmic compartment. Therefore, the inventors evaluated whether g3p fusion proteins can be exploited for antibody library screening purposes using the APEx format. The high affinity anti-PA M18 scFv discussed above, the anti-digoxin/digoxigenin 26-10 scFv, and an anti-methamphetamine scFv (Meth) were cloned in frame to the N-terminus of g3p downstream from a lac promoter in phagemid pAK200, which is widely used for phage display purposes and utilizes a short variant of gene III for g3p display (Krebber et al., 1997). Following induction with IPTG, cells expressing scFv-g3p fusions were permeabilized by Tris-EDTA-lysozyme and labeled with the respective fluorescent antigens (FIG. 9). High fluorescence was obtained for all three scFvs only when incubated with their respective antigens. Significantly, the mean fluorescence intensity of the scFvs fused to the N-terminus of g3p was comparable to that obtained by fusion to the C-terminus of the NlpA anchor. The results in FIG. 9 demonstrate that: (i) large soluble domains can be tethered N-terminally to a membrane anchor; (ii) antibody fragments cloned into phagemids for display on filamentous phage can be readily analyzed by flow cytometry using the APEx format, and (iii) scFv antibodies can be anchored on the cytoplasmic membrane either as N- or C-terminal fusions without loss of antigen binding.

2. Discussion

The inventors have developed a allowing efficient selection of high affinity ligand-binding proteins, and particularly scFv antibodies, from combinatorial libraries. In one aspect, APEx is based on the anchoring of proteins to the outer side of the inner membrane, followed by disruption of the outer membrane prior to incubation with fluorescently labeled antigen and FC sorting. This strategy offers several advantages over previous bacterial periplasmic and surface display approaches: 1) by utilizing a fatty acylated anchor to retain the protein in the inner membrane, a fusion as short as 6 amino acids is all that was required for the successful display, potentially decreasing deleterious effects that larger fusions may impose; 2) the inner membrane lacks molecules such as LPS or other complex carbohydrates that can sterically interfere with large antigen binding to displayed antibody fragments; 3) the fusion must only traverse one membrane before it is displayed; 4) both N- and C-terminal fusion strategies can be employed; and 5) APEx can be used directly for proteins expressed from popular phage display vectors. This latter point is particularly important because it enables hybrid library screening strategies, in which clones from a phage panning experiment can be quantitatively analyzed or sorted further by flow cytometry without the need for any subcloning steps.

APEx can be employed for the detection of antigens ranging from small molecules (e.g. digoxigenin and methamphetamine <1 kDa) to phycoerythrin conjugates (240 kDa). In fact, the phycoerythrin conjugate employed in FIG. 3B is not meant to define an upper limit for antigen detection, as it is contemplated that larger proteins may be used as well.

In the example, genes encoding scFvs that bind the fluorescently labeled antigen, were rescued from the sorted cells by PCR. An advantage of this approach is that it enables the isolation of clones that are no longer viable due to the combination of potential scFv toxicity, Tris-EDTA-lysozyme disruption, and FC shear forces. In this way, diversity of isolated clones is maximized. Yet another advantage of PCR rescue is that the amplification of DNA from pooled cells can be carried out under mutagenic conditions prior to subcloning. Thus, following each round of selection random mutations can be introduced into the isolated genes, simplifying further rounds of directed evolution (Hanes and Pluckthun, 1997). Further, PCR conditions that favor template switching among the protein encoding genes in the pool may be employed during the amplification step to allow recombination among the selected clones. It is likely that PCR rescue would be advantageous in other library screening formats as well.

An important issue with any library screening technology is the ability to express isolated clones at a high level. Existing display formats involve fusion to large anchoring sequences which can influence the expression characteristics of the displayed proteins. For this reason, scFvs that display well may not necessarily be amenable to high expression in soluble form as non-fusion proteins (Hayhurst et al., 2003). In contrast, the short (6 amino acid) tail that may be used for N-terminal tethering of proteins onto the cytoplasmic membrane in the current invention is unlikely to affect the expression characteristics of the fusion. Consistent with this hypothesis, all three affinity enhanced clones to the anthrax PA toxin isolated by APEx exhibited excellent soluble expression characteristics despite having numerous amino acid substitutions. Similarly, well-expressing clones have been obtained in the affinity maturation of a methamphetamine antibody, suggesting that the isolation of clones that can readily be produced in soluble form in bacteria at a large scale might be an intrinsic feature of selections with the invention.

In this example, the inventors employed APEx for affinity maturation purposes and have engineered scFvs to the B. anthracis protective antigen exhibiting K_{D} values as low as 21 pM. The scFv binding site exhibiting the highest affinity for PA has been humanized, converted to full length IgG and its neutralizing potential to anthrax intoxication is being evaluated in preclinical studies. In addition to affinity maturation, APEx can be exploited for several other protein engineering applications including the analysis of membrane protein topology, whereby a scFv antibody anchored in a periplasmic loop is able to bind fluorescent antigen and serves as a fluorescent reporter, and also, the selection of enzyme variants with enhanced function. Notably, APEx can be readily adapted to enzyme library sorting, as the cell envelope provides sites for retention of enzymatic catalytic products, thereby enabling selection based directly on catalytic turnover (Olsen et al., 2000). The inventors are also evaluating the utilization of APEx for the screening of ligands to membrane proteins. In conclusion, it has been demonstrated that anchored periplasmic expression has the potential to facilitate combinatorial library screening and other protein engineering applications.

E. Materials and Methods

1. Recombinant DNA Techniques

The leader peptide and first six amino acids of the mature NlpA protein flanked by NdeI and SfiI sites was amplified by whole cell PCR of XL1-Blue (Stratagene, CA) using primers

| | |
|---|---|
| BRH#08 | |
| 5′-GAAGGAGATATACATATGAAACTGACAACACATC | (SEQ ID NO:6) |
| ATCTA-3′ | |
| and | |
| BRH#09 | |
| 5′-CTGGGCCATGGCCGGCTGGGCCTCGCTGCTACTC | (SEQ ID NO:7) |
| TGGTCGCAACC-3′. | |

The resulting NlpA fragment was used to replace the pelB leader sequence of pMoPac1 (Hayhurst et al., 2003) via NdeI and SfiI to generate pAPEx1. scFv specific for digoxin (Chen et al., 1999), Bacillus anthracis protective antigen PA (Maynard et al., 2002) and methamphetamine were inserted downstream of the NlpA fragment in pAPEx1 via the non-compatible SfiI sites. Corresponding g3p fusions of the scFv were made by cloning the same genes into phage display vector pAK200 (Krebber et al., 1997).
2. Growth Conditions

E. coli ABLE C™ (Stratagene) was the host strain used throughout. E. coli transformed with the pAPEx1 or pAK200 derivatives were inoculated in terrific broth (TB) supplemented with 2% glucose and chloramphenicol at 30 ug/ml to an OD600 of 0.1. Cell growth and induction were performed as described previously (Chen et al., 2001). Following induction, the cellular outer membrane was permeabilized as described (Neu and Heppel, 1965). Briefly, cells (equivalent to approx 1 ml of 20 OD600) were pelleted and resuspended in 350 μl of ice-cold solution of 0.75M sucrose, 0.1M Tris-HCl pH8.0, 100 μg/ml hen egg lysozyme. 700 μl of ice-cold 1 mM EDTA was gently added and the suspension left on ice for 10 min. 50 μl of 0.5M MgCl₂ was added and the mix left on ice for a further 10 min. The resulting cells were gently pelleted and resuspended in phosphate buffered saline (1×PBS) with 200 nM probe at room temperature for 45 min, before evaluation by FC.

3. Fluorescent Probe

The synthesis of digoxigenin-BODIPY has been described previously (Daugherty et al., 1999). Methamphetamine-fluorescein conjugate was a gift from Roche Diagnostics. Purified PA protein kindly provided by S. Leppla NIH, was conjugated to BODIPY™ at a 1 to 7 molar ratio with bodipy FL SE D-2184 according to the manufacturers instructions. Unconjugated BODIPY™ was removed by dialysis.

To synthesize digoxigenin-phycoerythrin, R-phycoerythrin and 3-amino-3-dioxydigxigenin hemisuccinamide, succinimidyl ester (Molecular Probes) were conjugated at a 1 to 5 molar ratio according to the manufacturers instructions. Free digoxigenin was removed by dialysis in excess PBS.

4. Affinity Maturation of scFv Libraries with FC

Libraries were made from the 14B7 parental scFv using error prone PCR using standard techniques (Fromant et al., 1995) and cloned into the pAPEx1 expression vector. Upon transformation, induction and labeling the cells were then stained with propidium iodide (PI emission 617 nm) to monitor inner membrane integrity. Cells were analyzed on a MoFlo (Cytomation) droplet deflection flow cytometer using 488 nm Argon laser for excitation. Cells were selected based on improved fluorescence in the Fluorescein/Bodipy FL emission spectrum detecting through a 530/40 band pass filter and for the absence of labeling in PI emission detecting through a 630/40 band pass filter.

E. coli captured after the first sort were immediately resorted through the flow cytometer. Subsequently, the scFv genes in the sorted cell suspension were amplified by PCR. Once amplified, the mutant scFv genes were then recloned into pAPEx1 vector, retransformed into cells and then grown overnight on agar plates at 30° C. The resulting clones were subjected to a second round of sorting plus resorting as above, before scFv genes were subcloned into pMoPac16 (Hayhurst et al., 2003) for expression of scAb protein.

5. Surface Plasmon Resonance Analysis

Monomeric scAb proteins were purified by IMAC/size-exclusion FPLC as described previously (Hayhurst et al., 2003). Affinity measurements were obtained via SPR using a BIACore3000 instrument. Approximately 500 RUs of PA was coupled to a CM5 chip using EDC/NHS chemistry. BSA was similarly coupled and used for in line subtraction. Kinetic analysis was performed at 25° C. in BIA HBS-EP buffer at a flow rate 100 μl/min. Five two fold dilutions of each antibody beginning at 20 nM were analyzed in triplicate.

### Example 7

### Construction of Vectors for the Co-Expression of Anchored Binding Protein and Ligands

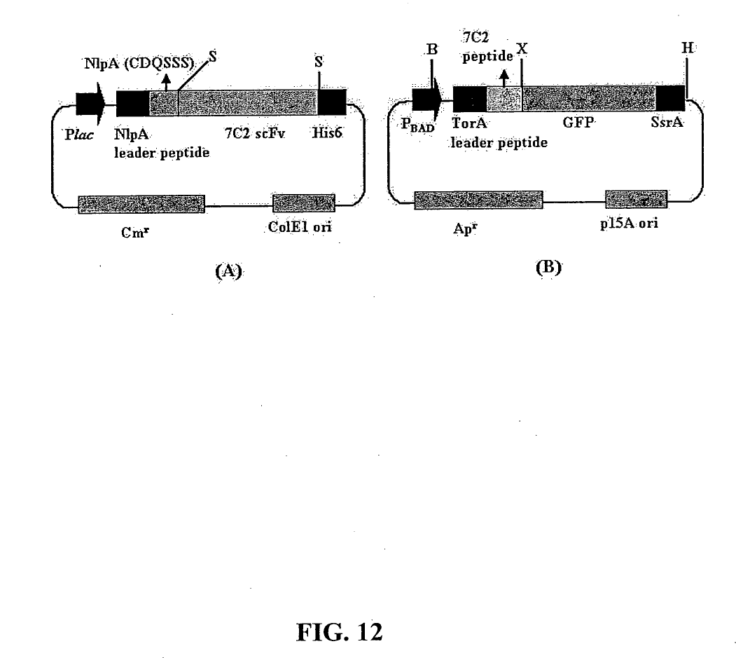

A 7C2-scFv coding sequence, which recognizes the peptide antigen 7C2 from the MacI protein with a K_{D}=142 nM, was obtained from MorphoSysAG (Germany) and cloned into an SfiI site of NlpA-[Dig scFv] expressing vector (FIG. 12A). In this construct, 7C2 scFv can be expressed in periplasm, tethered to the inner membrane of E. coli via lipidation of a small N-terminal 6 amino acid (CDQSSS) (SEQ ID NO:26) fusion of NlpA, non-essential E. coli lipoprotein.

For the construction of vector pTGS30, plasmid pTGS (DeLisa et al., 2002), which contains a BAD promoter and TorA-GFP-SsrA expression cassette, was digested by BamHI and HindIII restriction enzymes and the fragment cloned into plasmid pBAD30 (Guzman et al., 1995) containing an Ap resistance gene. In this construct (pTGS30), only mature GFP protein was produced in the periplasm by the Twin-Arginine Translocation (TAT) pathway. Plasmid pT7C2GS30 was constructed by overlapping PCR using the primers BAD-F (5′-AGCGGATCCTACCTGACGC-3′) (SEQ ID NO:27), 7C2-R1 (5′-CCTTGAAGGTGAAACAAGCGTCAGTCGCCGCTTGCGC-3′) (SEQ ID NO:28), 7C2-R2 (5′-GTTCGGATTGTTTTGAAATTCCTTGAAGGTGAAACAAGCG-3′) (SEQ ID NO:29), 7C2-R3 (5′-CTTTACCAGAGAACGCGGGTTCGGATTGTTTTGAAATTCC-3′) (SEQ ID NO:30) and 7C2-R4 (5′-CGTCTAGATCCACCCTTTACCAGAGAACGCGGG-3′) (SEQ ID NO:31) with pTGS30 as template DNA to introduce the sequence encoding the 7C2 peptide (CFTFKEFQNNPNPRSLVK) (SEQ ID NO:32) to the C-terminal of TorA leader sequence. PCR product was digested with BamHI and XbaI and cloned into plasmid pTGS30, digested by same restriction enzymes. In this construct (pT7C2GS30, FIG. 12B), a 7C2 peptide fused GFP protein was produced and folded in the cytoplasm and then transported into the periplasm by the TAT pathway. Cytoplasmic GFP fusion protein was degraded by a protease which recognizes SsrA peptide at the C-terminus of the fusion protein.

### Example 8

### Selection of Cells Co-Expressing Ligands and Binding Proteins by APEx

Overnight cultures of XL1-Blue cells were subcultured into fresh TB medium at 37° C. and induced with 0.2% arabinose for the expression of 7C2 peptide-GFP fusion protein and 0.2 mM IPTG for the expression of 7C2 scFv-APEx in mid-exponential phase growth to yield expression of the 7C2 peptide-GFP fusion protein and 7C2 scFv-APEx, respectively. After 4 hr, cells were collected and spheroplasts were prepared by lysozyme-EDTA treatment to remove the unbound GFP fused probe in the periplasm. Specifically, the collected cells were resuspended in a buffer (350 μL) containing 0.1 M Tris-Cl (pH 8.0) and 0.75 M sucrose, and then 700 μL of 1 mM NaEDTA was added. Lysozyme (Sigma) was added to 100 μg/mL and cells were incubated at room temp for 20 min. Finally, 50 μL of 0.5 M MgCl₂ was added and further incubated on ice for 10 min. The spheroplasted cells were pelleted by 10 min of centrifugation at 10,000 rpm and then resuspended in 1×PBS buffer. 5 μL of resuspended cells were diluted into 2 mL of 1×PBS buffer prior to analysis using a BD FACSort from BD Biosciences.

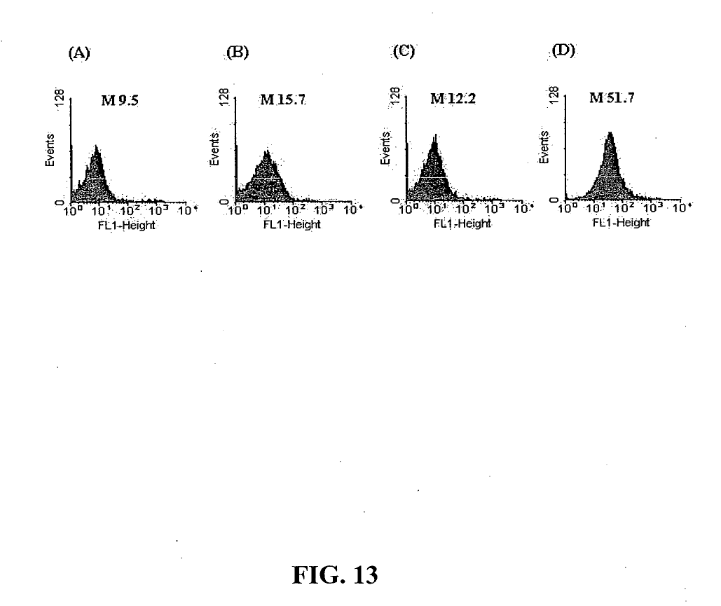

As shown in FIG. 13, GFP-peptide coexpressed with 7C2 anti-peptide scFv-APEx (FIG. 13D) exhibited a 4-fold higher fluorescence compared to the other control cells expressing either: (FIG. 13A) GFP-peptide fusion alone, (FIG. 13B) GFP-peptide co-expressed with an NlpA-fused irrelevant scFv (26-10 scFv) or (FIG. 13C) GFP without peptide antigen co-expressed with an 7C2 scFv-APEx. This data indicates that the GFP-peptide was bound to 7C2 scFv tethered to the inner membrane, and was detected successfully by FACS. Additionally, the use of 26-10 scFv-APEx instead of 7C2 scFv-APEx resulted in the loss of fluorescence, which demonstrates the high specificity of this method. The results confirm the ability to select cells that co-express a target ligand and candidate binding protein having affinity for the target ligand using APEx.

### Example 9

### Selection of Cells Co-Expressing Ligands and Binding Proteins by APEX Using a Peptide Label-Specific Antibody Pair to Detect the Interaction: Construction of Vectors

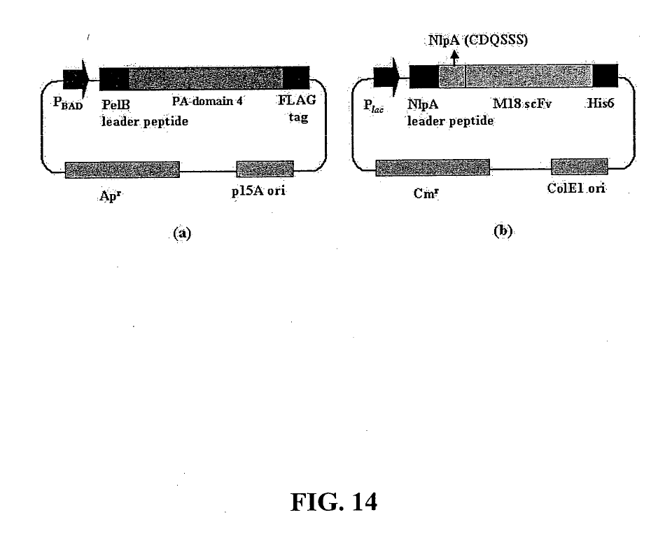

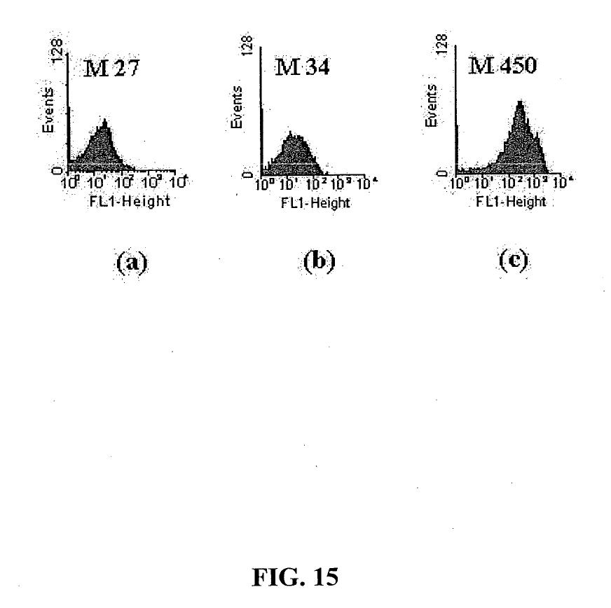

An M18-scFv coding sequence (SEQ ID NO:23) was cloned into the SfiI site of the NlpA-[Dig scFv] expression vector. In this construct (pM18APEx) (FIG. 14B), M18 scFv can be expressed in the periplasm and tethered to the inner membrane of E. coli via lipidation of a small N-terminal 6 amino acid (CDQSSS) (SEQ IN NO:26) fusion of NlpA, non-essential E. coli lipoprotein.

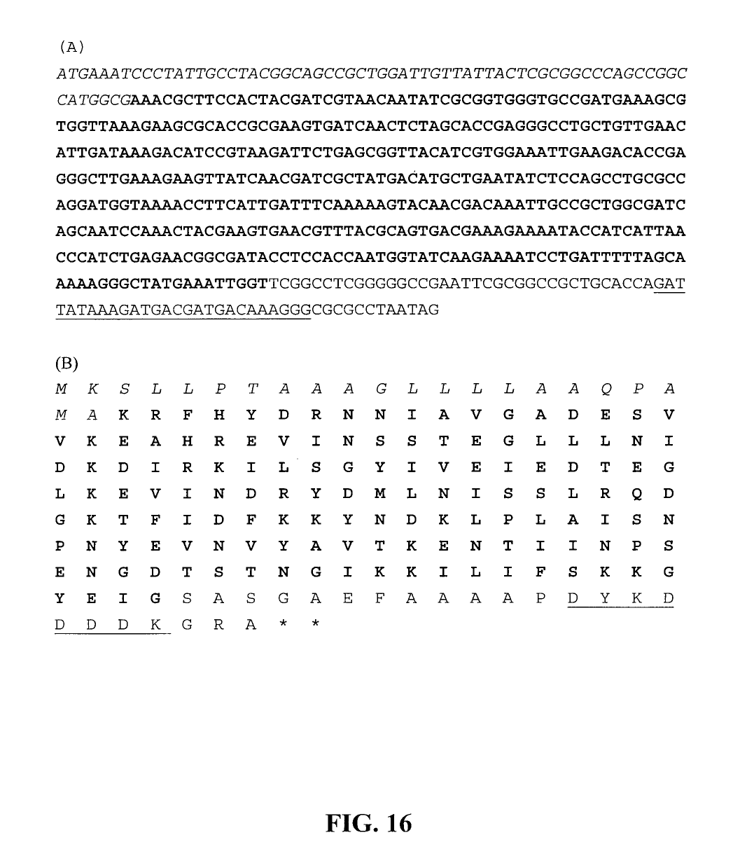

Bacillus anthracis Protective Antigen (PA) consists of 4 domains. It is known that domain 4 coding sequence (residues 596-735) is responsible for the affinity of the PA antibody. The domain 4 coding sequence was synthesized by overlapping PCR using 13 primers. These primers sequences are listed in Table 1. The PCR product (PA-domain 4) was then digested with the SfiI restriction enzyme and cloned into pMoPac16, which is a vector containing the PelB leader peptide. In the resulting construct (pPelBPAD4), PA-Domain4 is fused to C-terminal of PelB so that the fusion protein can be secreted into the periplasm. To fuse the FLAG tag (DYKDDDDK) (SEQ ID NO:33) to the C-terminus of PA-Domain 4, PCR was done using template DNA pPelBPAD4 and the three primers MoPac-Sac-F1 (GTCGAGCTCAGAGAAGGAGATATACATATG) (SEQ ID NO:34), PAD4-Hind-R1 (CTTTGTCATCGTCATCTTTATAATCTGGTGCAGCGGCCGCGAATTCGG) (SEQ ID NO:35), PAD4-Hind-R2 (CGAAGCTTCTATTAGGCGCGCCCTTTGTCATCGTCATCTTTAT) (SEQ ID NO:36). The PCR product was digested with the restriction enzymes SacI and HindIII and cloned into pBAD30 (Guzman L M et al., J. Bacteriol. 177: 4121-4130 1995) following its digestion using the same restriction enzymes. In this construct (pB30PelBD4FL), the PelB leader peptide-PA-Domain4-FLAG tag fused gene expression was under the control of the arabinose induction promoter (BAD promoter). The pB30PelBD4FL construct also contains an ampicillin resistance gene as a selection marker as well as a low copy number origin of replication (p15A ori) (FIG. 14A). The sequence of the PA-domain 4 pB30PelBD4FL construct (SEQ ID NO:37 and SEQ ID NO:38) was confirmed by sequencing experiment (FIG. 16).

TABLE 1
List of primer and their sequences used for
synthesis of PA-Domain 4.

| | |
|---|---|
| Primer Name | Sequences (5′ → 3′) |

| | |
|---|---|
| PA-D4-F1 | GATCGCTATGACATGCTGAATATCTCCAGCCTGCGCCAG |
| | GATGGTAAAAC (SEQ ID NO:39) |
| PA-D4-F2 | AGACACCGAGGGCTTGAAAGAAGTTATCAACGATCGCTA |
| | TGACATGCTG (SEQ ID NO:40) |
| PA-D4-F3 | GTAAGATTCTGAGCGGTTACATCGTGGAAATTGAAGACA |
| | CCGAGGGCTTG (SEQ ID NO:41) |
| PA-D4-F4 | GGCCTGCTGTTGAACATTGATAAAGACATCCGTAAGATT |
| | CTGAGCGGTTA (SEQ ID NO:42) |
| PA-D4-F5 | CGCACCGCGAAGTGATCAACTCTAGCACCGAGGGCCTGC |
| | TGTTGAACATT (SEQ ID NO:43) |
| PA-D4-F6 | GTGGGTGCCGATGAAAGCGTGGTTAAAGAAGCGCACCGC |
| | GAAGTGATCA (SEQ ID NO:44) |
| PA-D4-F7 | AAACGCTTCCACTACGATCGTAACAATATCGCGGTGGGT |
| | GCCGATGAAAG (SEQ ID NO:45) |
| PA-D4-F8 | GCTAGGCCCAGCCGGCCATGGCGAAACGCTTCCACTACG |
| | ATC (SEQ ID NO:46) |
| PA-D4-R1 | TTTGTCGTTGTACTTTTTGAAATCAATGAAGGTTTTACC |
| | ATCCTGGCGC (SEQ ID NO:47) |
| PA-D4-R2 | TAGTTTGGATTGCTGATATACAGCGGCAATTTGTCGTTG |
| | TACTTTTTGA (SEQ ID NO:48) |
| PA-D4-R3 | TTCTTTCGTCACTGCGTAAACGTTCACTTTGTAGTTTGG |
| | ATTGCTGATAT (SEQ ID NO:49) |
| PA-D4-R4 | GCCGTTCTCAGATGGGTTAATGATGGTATTTTCTTTCGT |
| | CACTGCGTAA (SEQ ID NO:50) |
| PA-D4-R5 | CAGGATTTTCTTGATACCATTGGTGGAGGTATCGCCGTT |
| | CTCAGATGGG (SEQ ID NO:51) |
| PA-D4-R6 | ACCAATTTCATAGCCCTTTTTGCTAAAAATCAGGATTTT |
| | CTTGATACCAT (SEQ ID NO:52) |
| PA-D4-R7 | GCTAGGCCCCCGAGGCCGAACCAATTTCATAGCCCTTTT |
| | TGC (SEQ ID NO:53) |

### Example 10

### Selection of Cells Co-Expressing Ligands and Binding Proteins by APEX Using a Peptide Label-Specific Antibody Pair to Detect the Interaction: Analysis of Fluorescence

The two plasmids (pB30PelBD4FL and pM18APEx) were transformed into E. coli Jude1 cells. Overnight cultures of the resulting cells were then subcultured into fresh TB medium at 37° C. After 2 hr, the flask was moved to a 25° C. shaking water bath to decrease the culture temperature. After 30 min cooling at 25° C., induction was done with 0.2% arabinose for the expression of PelB-PA-Domain4-FLAG tag fusion protein and 1 mM IPTG for the expression of M18 scFv-APEx to yield expression of the PelB-PA-Domain4-FLAG tag fusion protein and M18 scFv-APEx, respectively. After 4 hr, cells were collected and spheroplasts were prepared by lysozyme-EDTA treatment to remove the unbound PA-Domain4-FLAG tag probe from the periplasm. Specifically, the collected cells were resuspended in a buffer (350 μL) containing 0.1 M Tris-Cl (pH 8.0) and 0.75 M sucrose, and then 700 μL of 1 mM NaEDTA was added. Lysozyme (Sigma) was added to 100 μg/mL and cells were incubated at room temperature for 10 min. Finally, 50 μL of 0.5 M MgCl₂ was added and further incubated on ice for 10 min. The spheroplast cells were pelleted by 10 min of centrifugation at 10,000 rpm and then resuspended in 1×PBS buffer (phosphate buffered saline). For flow cytometric analysis, 0.1 mL of spheroplast cells were mixed with 100 nM of anti-FLAG Ab (M2)-FITC conjugate probe (Sigma) in 0.9 mL of 1×PBS and after 30 min of incubation at room temperature with shaking, the cells were collected by centrifugation. Under this procedure, if the PA-Domain4-FLAG protein probe binds to M18 scFv tethered to inner membrane, the FLAG tag would become labeled with anti-FLAG Ab (M2)-FITC conjugate probe. The cells were resuspended in 1 mL of 1×PBS and a 5 μL aliquot was diluted into 2 mL of 1×PBS buffer prior to analysis using a BD FACSort (BD Biosciences).

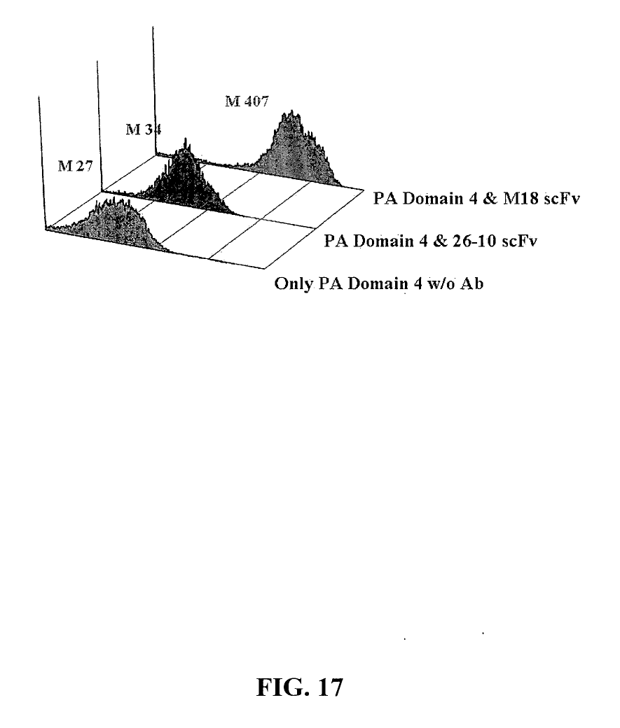

As shown in FIG. 17, cells with PA-Domain4-FLAG protein co-expressed with M18 scFv-APEx exhibited a 15-fold higher fluorescence compared to the other control cells expressing either: PA-Domain4-FLAG protein alone (pB30PelBD4FL) or co-expressed with an NlpA-fused irrelevant scFv (26-10 scFv & pB30PelBD4FL). This data indicates that the PA-Domain4-FLAG protein was bound to the M18 scFv tethered to the inner membrane, and was successfully detected by FACS after labeling with anti-FLAG Ab-FITC conjugate probe. The 15-fold lower fluorescence of cells expressing 26-10 scFv-APEx instead of M18 scFv-APEx demonstrated the high specificity of the method. The results confirmed the ability to select cells that co-express a target ligand and a binding protein using a peptide label-specific antibody pair by APEx. From these results, it can be concluded that ligand-anchored protein hybridization works well and is useful for identification of protein-protein interactions.

### Example 11

### Examination of the High Selectivity of Co-Expression of Mutated Ligand Protein

Previously, Rosovitz et al. reported that the Tyr at position 681 of the PA protein is responsible for PA toxicity yet has no effect on the Ab binding, and that the Tyr at position 688 position is critical for the Ab binding, so the change of this residue to other amino acids can cause the loss of Ab binding (Rosovitz et al., J. Biol. Chem 278:30936 2003). To verify the high specificity of this system, two mutants of PA Domain4 were constructed. In one mutant, Y681A, the Tyrosine at the 681 position was changed to alanine. In a second mutant, Y688A, the Tyrosine at the 688 position was changed to alanine.

For the construction of mutant Y681A, the two primers Y681-F1 (CAAAAAGGCGAACGACAAATTGCCGCTGT) (SEQ ID NO:54) and Y681-R1 (CAATTTGTCGTTCGCCTTTTTGAAATCAATGAAGGTTT) (SEQ ID NO:55) were synthesized. Two PCR reactions were then performed using pB30PelBD4FL as template DNA, the first PCR with the two primers MoPac-Sac-F1 (SEQ ID NO:34) and Y681-R1 (SEQ ID NO:55), and the second PCR with the two primers PAD4-Hind-R2 (SEQ ID NO:36) and Y681-F1 (SEQ ID NO:54). Each PCR product was then purified and mixed and overlapping PCR was done with the two primers MoPac-Sac-F1 (SEQ ID NO:34) and PAD4-Hind-R2 (SEQ ID NO:36). After overlapping PCR was complete, the PCR product was digested with the two restriction enzymes SacI and HindIII and then cloned into pBAD30. In the resulting plasmid (pB30D4Y681AFL) the mutation point (Y681A) was confirmed by a sequencing experiment.

For the construction of mutant Y688A, the two primers Y688-F1 (TTGCCGCTGGCGATCAGCAATCCAAACTACAAAG) (SEQ ID NO:56) and Y688-R1 (GCTGATCGCCAGCGGCAATTTGTCGTTG) (SEQ ID NO:57) were synthesized. Two PCR reactions were then performed using pB30PelBD4FL as template DNA, the first PCR with the two primers MoPac-Sac-F1 (SEQ ID NO:34) and Y688-R1 (SEQ ID NO:57), and the second PCR with the two primers PAD4-Hind-R2 (SEQ ID NO:36) and Y688-F1 (SEQ ID NO:56). Each PCR product was then purified and mixed and overlapping PCR was done with the two primers MoPac-Sac-F1 (SEQ ID NO:34) and PAD4-Hind-R2 (SEQ ID NO:36). After overlapping PCR was complete, the PCR product was digested with the two restriction enzymes SacI and HindIII and then cloned into pBAD30. In the resulting plasmid (pB30D4Y688AFL) the mutation point (Y688A) was confirmed by sequencing experiment.

Each plasmid (pB30D4Y681AFL and pB30D4Y688AFL) was then separately transformed into E. coli Jude1 cells containing pM18scFv-APEx. The resulting cells were cultured, induced, spheroplasted, and then labeled with the anti-FLAG-Ab-FITC conjugate using techniques described in the previous example. The cell were then analyzed using a BD FACSort (BD Biosciences).

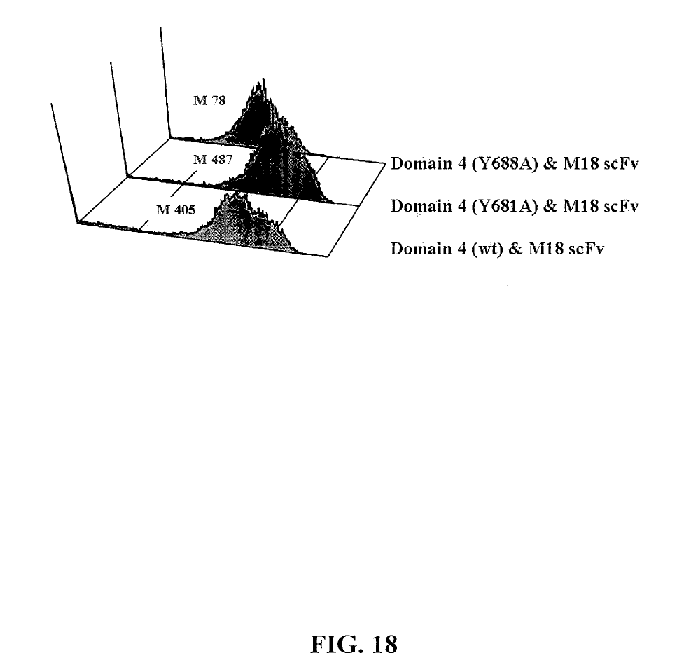

As shown in FIG. 18, cells with PA-Domain4-Y688A-FLAG protein co-expressed with M18 scFv-APEx exhibited a 6-fold lower fluorescence compared to the other control cells expressing either: PA-Domain4-FLAG protein co-expressed with M18 scFv-APEx and PA-Domain4-Y681A-FLAG protein coexpressed with M18 scFv-APEx. This data indicated that the co-expression approach has a high selectivity sufficient to distinguish even a single amino acid mutation.

### Example 12

### Examination of One Plasmid System for the Co-Expression of Anchored Binding Protein and Ligand Protein

In the three previous examples (Examples 9, 10 and 11), two plasmids were used for co-expression of ligand protein and anchored binding protein. A one plasmid system was also analyzed for the expression of both the ligand protein and the anchored binding protein from a single plasmid.

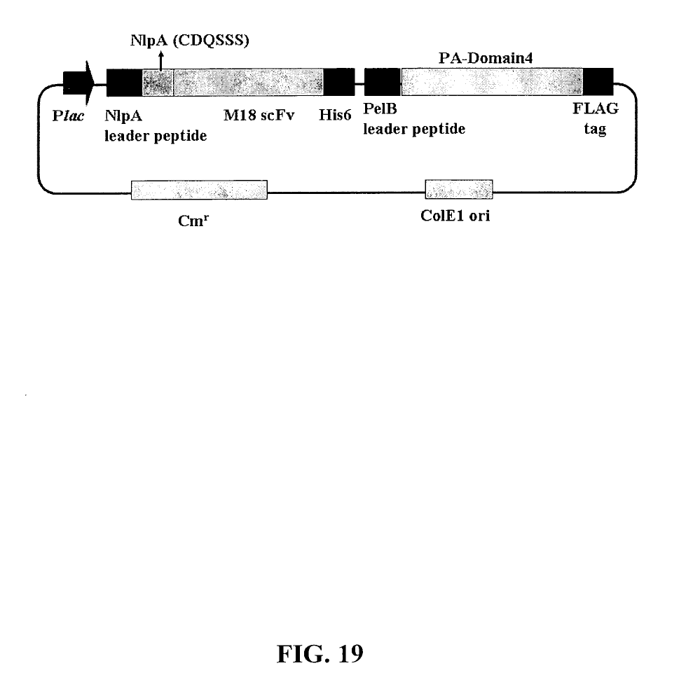

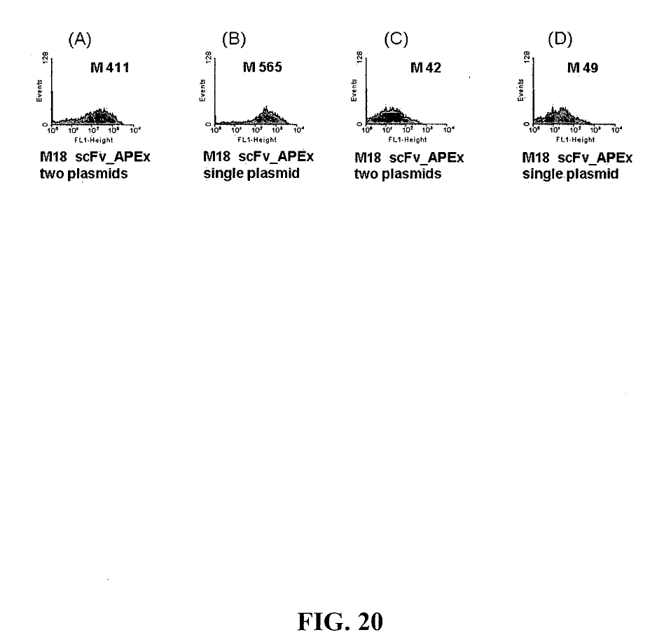

For the construction of the one plasmid system, two PCR primers, D4-Hin-F1 (GCAAGCTTAGAGAAGGAGATATACATATGAAATC) (SEQ ID NO:58), and D4-Hin-R1 (CCAAGCTTCTATTAGGCGCGCCCTTTG) (SEQ ID NO:59) were synthesized. A PCR reaction was then performed using the two primers and pB30PelBD4FL as a template. The PCR product was digested with HindIII restriction enzyme and cloned into a pM18 scFv-APEx vector previously digested with HindIII restriction enzyme and dephosphorylated with CIP. The resulting plasmid (pM18 scFv-D4) contained the M18 scFv APEx and PelB-PA-Domain4-FLAG tag expression system under the control of a single inducible promoter (lac promoter) (FIG. 19). Also, the Y688 mutant of Domain4 was amplified with same PCR primers (D4-Hin-F1 and D4-Hin-R1) and cloned into same pM18 scFv-APEx resulting in pM18 scFv-D4Y688. Each plasmid was then separately transformed into E. Coli Jude1 cells. The resulting cells were cultured, induced, and spheroplasted using techniques described in the previous example, except that for the expression of both genes (M18 scFv and Domain 4-wild type or Y688A mutant), only one inducer (IPTG) was used. The cells were then labeled with the anti-FLAG-Ab-FITC conjugate as described in the previous example and were then analyzed using a BD FACSort (BD Biosciences).

As shown in FIG. 20, the one plasmid system showed a slightly higher fluorescence than the two plasmid system for co-expression of wild type domain 4 and M18 scFv (FIGS. 20A and 20B). In the co-expression of the Y688A mutant of domain 4 and M18 scFv, the one plasmid system showed a low fluorescence similar to that of the two plasmid system (FIGS. 20C and 20D). This data indicated that the one plasmid system can distinguish positive fluorescence clones in FACS sorting. These results show an enhanced ability in this example for the one plasmid system as compared to the two plasmid system for the selection of cells that co-express a target ligand and candidate binding protein having affinity for the target ligand using APEx.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Abbondanzo et al., Am J Pediatr Hematol Oncol, 12 (4):480-9, 1990.
Almendro et al., J Immunol. 157:5411, 1996.
Angel et al., Cell, 49:729, 1987b.
Angel et al., Mol. Cell. Biol., 7:2256, 1987a.
Atchison and Perry, Cell, 46:253, 1986.
Atchison and Perry, Cell, 48:121, 1987.
Atherton et al., Biol. of Reproduction, 32:155, 1985.
Banerji et al., Cell, 27:299, 1981.
Banerji et al., Cell, 35:729, 1983.
Barbas et al., Proc. Natl. Acad. Sci. USA, 88:7978-7982, 1991.
Bellus, J. Macromol. Sci. Pure Appl. Chem., RS3241 (1):1355-1376, 1994.
Berberian et al., Science, 261:1588-1591, 1993.
Berkhout et al., Cell, 59:273, 1989.
Berrier et al., J. Bacteriol., 182:248, 2000.
Blanar et al, EMBO J., 8:1139, 1989.
Boder and Wittrup, Methods Enzymol., 328:430-444, 2000.
Boder et al., Proc. Natl. Acad. Sci. USA, 97:10701-10705, 2000.
Bodine and Ley, EMBO J., 6:2997, 1987.
Boeke and Model, Proc. Natl. Acad. Sci. USA, 79:5200-5204, 1982.
Boeke et al., Mol. Gen. Genet., 186: 1982.
Boshart et al., Cell, 41:521, 1985.
Bosze et al., EMBO J., 5:1615, 1986.
Braddock et al., Cell, 58:269, 1989.
Bukau et al., J. Bacteriol., 163:61, 1985.
Bulla and Siddiqui, J. Virol., 62:1437, 1986.
Burioni et al., Res. Virol., 149:327, 1998.
Burman et al., J. Bacteriol., 112:1364, 1972.
Campbell and Villarreal, Mol. Cell. Biol., 8:1993, 1988.
Campere and Tilghman, Genes and Dev., 3:537, 1989.
Campo et al., Nature, 303:77, 1983.
Carter et al., Nucleic Acids Res 13:4431, 1985.
Celander and Haseltine, J. Virology, 61:269, 1987.
Celander et al., J. Virology, 62:1314, 1988.
Chandler et al., Cell, 33:489, 1983.
Chang et al., Mol. Cell. Biol., 9:2153, 1989.
Chatterjee et al., Proc. Nat'l Acad. Sci. USA., 86:9114, 1989.
Chen et al., J. Mol. Biol., 293:865, 1999.
Chen et al., Nat. Biotechnol., 19:537-542, 2001.
Chen et al., Protein Eng., 12:349-356, 1999.
Choi et al., Cell, 53:519, 1988.
Chowdhury and Pastan, Nat. Biotech., 17:568, 1999.
Cleary et al., Trends Microbiol., 4:131-136, 1994.
Coffin, In: Virology, ed., New York: Raven Press, pp. 1437-1500, 1990.
Cohen et al., Proc. Nat'l Acad. Sci. USA 75:472, 1987.
Coia et al., Gene 201:203, 1997.
Corey et al., Gene, 128:129, 1993.
Costa et al., Mol. Cell. Biol., 8:81, 1988.
Cripe et al., EMBO J., 6:3745, 1987.
Culotta and Hamer, Mol. Cell. Biol., 9:1376, 1989.
Dall'Acqua and Carter, Curr. Opin. Struct. Biol., 8:443, 1998.
Dandolo et al., J. Virology, 47:55, 1983.
Daugherty et al., J. Immunol. Methods. 243:211, 2000.
Daugherty et al., Proc. Natl. Acad. Sci. USA, 97:2029-2034, 2000.
Daugherty et al., Prot. Eng., 11:825, 1998.
Daugherty et al., Protein Eng., 12:613-621, 1999.
De Haard et al., J. Biol. Chem., 274:18218, 1999.
De Jager R et al., Semin Nucl Med 23:165, 1993.
De Villiers et al., Nature, 312:242, 1984.
De Wildt et al., Nat. Biotechnol. 18:, 989, 2000.
Decad and Nikaido, J. Bacteriol., 128:325, 1976.
DeLisa M P, Samuelson P, Palmer T, Georgiou G. Genetic analysis of the twin arginine translocator secretion pathway in bacteria. J Biol. Chem. (2002) 277 (33):29825-29831.
Deng et al., J. Biol. Chem., 269:9533, 1994.
Deng et al., Proc. Natl. Acad. Sci. USA. 92:4992, 1995.
Deschamps et al., Science, 230:1174, 1985.
Dholakia et al., J. Biol. Chem., 264, 20638-20642, 1989.
Doolittle M H and Ben-Zeev O, Methods Mol. Biol., 109:215, 1999.
Duenas and Borrebaeck, Biotechnology, 12:999, 1994.
Edbrooke et al., Mol. Cell. Biol., 9:1908, 1989.
Edlund et al., Science, 230:912, 1985.
Farmer et al., FEMS Microbiol. Lett., 176:11, 1999.
Feldhaus et al., Nat. Biotechnol., 21:163-170, 2003.
Feng and Holland, Nature, 334:6178, 1988.
Firak and Subramanian, Mol. Cell. Biol., 6:3667, 1986.
Frohman, In: PCR PROTOCOLS: A GUIDE TO METHODS AND APPLICATIONS, Academic Press, N.Y., 1990.
Fromant et al., Anal. Biochem., 224:347-353, 1995.
Fujita et al., Cell, 49:357, 1987.
Gennity and Inouye J. Bacteriol 174 (7):2095, 1992
Georgiou et al., Nat. Biotechnol. 15:29, 1997.
Georgiou, Adv. Protein Chem., 55:293-315, 2000.
Gilles et al., Cell, 33:717, 1983.
Gloss et al., EMBO J., 6:3735, 1987.
Godbout et al., Mol. Cell. Biol., 8:1169, 1988.
Goodboum and Maniatis, Proc. Nat'l Acad. Sci. USA, 85:1447, 1988.
Goodbourn et al., Cell, 45:601, 1986.
Gough et al., J. Immunol. Met., 228:97, 1999.
Greene et al., Immunology Today, 10:272, 1989.
Griep et al., Prot. Exp. Purif., 16:63, 1999.
Griffiths et al., EMBO J., 13: 3245, 1994.
Grosschedl and Baltimore, Cell, 41:885, 1985.
Gulbis and Galand, Hum Pathol 24:1271, 1993.
Guzman L M, Belin D, Carson M J, Beckwith J. Tight regulation, modulation, and high-level expression by vectors containing the arabinose PBAD promoter. J. Bacteriol. (1995) 177 (14):4121-4130.
Hanes and Pluckthun, Proc. Natl. Acad. Sci. USA, 94:4937-4942, 1997.
Haslinger and Karin, Proc. Nat'l Acad. Sci. USA., 82:8572, 1985.
Hauber and Cullen, J. Virology, 62:673, 1988.
Hawkins et al., J. Mol. Biol., 226:889, 1992.
Hayhurst and Georgiou, Curr. Opin. Chem. Biol., 5:683-689, 2001.
Hayhurst and Harris, Protein Expr. Purif., 15:336-343, 1999.
Hayhurst et al., J. Immunol. Methods, 276:185-196, 2003.
Hayhurst, Protein Expr. Purif., 18:1-10, 2000.
Hearing et al., J. Virol., 67:2555-2558, 1987.
Hen et al., Nature, 321:249, 1986.
Hensel et al., Lymphokine Res., 8:347, 1989.
Herr and Clarke, Cell, 45:461, 1986.
Hirochika et al., J. Virol., 61:2599, 1987.
Hirsch et al., Mol. Cell. Biol., 10: 1959, 1990.
Hobot et al., J. Bacteriol. 160:143, 1984.
Hoess, Chem. Rev., 101:3205-3218, 2001.
Holbrook et al., Virology, 157:211, 1987.
Hoogenboom et al., Adv. Drug. Deliv. Rev., 31:5, 1998.
Horlick and Benfield, Mol. Cell. Biol., 9:2396, 1989.
Hsiung et al, Biotechnology, 4:991, 1994.
Huang et al., Cell, 27:245, 1981.
Hudson and Souriau, Nat. Med. 9:129-134, 2003.
Hudson, Curr. Opin. Biotechnol., 9:395, 1998.
Hultgren et al., Bacterial Adhesins Assembly, Vol. 2., 1996.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Innis et al., Proc Natl Acad Sci USA. 85:9436, 1988.
Irvin et al., J. Bacteriol., 145:1397, 1981.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Jeffrey et al., Proc. Natl. Acad. Sci. USA. 90:10310, 1993.
Johns et al., J. Immunol. Methods, 239:137, 2000.
Johnson et al., Mol. Cell. Biol., 9:3393, 1989.
Jouenne and Junter, FEMS Microbiol. Lett., 56:313, 1990.
Kadesch and Berg, Mol. Cell. Biol., 6:2593, 1986.
Kang et al., Science, 240:1034-1036, 1988.
Karin et al., Mol. Cell. Biol., 7:606, 1987.
Katinka et al., Cell, 20:393, 1980.
Katinka et al., Nature, 290:720, 1981.
Kawamoto et al., Mol. Cell. Biol., 8:267, 1988.
Khatoon et al., Ann. of Neurology, 26, 210-219, 1989.
Kiledjian et al., Mol. Cell. Biol., 8:145, 1988.
King et al., J. Biol. Chem., 269:10218, 1989.
Kjaer et al., FEBS Lett., 431:448, 1998.
Klamut et al., Mol. Cell. Biol., 10:193, 1990.
Knappick et al., J. Mol. Biol., 296:57, 2000.
Koch et al., Mol. Cell. Biol., 9:303, 1989.
Kohler et al., Methods Enzymol., 178:3, 1989.
Kraus et al., FEBS Lett., 428:165, 1998.
Krebber et al., Gene, 178:71, 1996.
Krebber et al., J. Immunol. Methods, 201:35-55, 1997.
Kreier et al., Infection, Resistance and Immunity, Harper & Row, New York, (1991)).
Kriegler and Botchan, In: Eukaryotic Viral Vectors, Y. Gluzman, ed., Cold Spring Harbor: Cold Spring Harbor Laboratory, NY, 1982.
Kriegler et al., Cell, 38:483, 1984a.
Kriegler et al., Cell, 53:45, 1988.
Kriegler et al., In: Cancer Cells 2/Oncogenes and Viral Genes, Van de Woude et al. eds, Cold Spring Harbor, Cold Spring Harbor Laboratory, 1984b.
Kriegler et al., In: Gene Expression, D. Hamer and M. Rosenberg, eds., New York: Alan R. Liss, 1983.
Kuhl et al., Cell, 50:1057, 1987.
Kunz et al., Nucl. Acids Res., 17:1121, 1989.
Kwoh et al, Proc Natl Acad Sci USA. 86:1173, 1989.
Labischinski et al., J. Bacteriol., 162:9, 1985.
Lareyre et al., J. Biol. Chem., 274:8282, 1999.
Larsen et al., Proc. Nat'l Acad. Sci. USA., 83:8283, 1986.
Laspia et al., Cell, 59:283, 1989.
Latimer et al., Mol. Cell. Biol., 10:760, 1990.
Lee et al., J Auton Nerv Syst. 74:86, 1997
Lee et al., Nature, 294:228, 1981.
Lenert et al., Science, 248:1639-1643, 1990.
Levinson et al., Nature, 295:79, 1982.
Levitan, J. Mol. Biol., 277:893, 1998.
Li et al., Nat. Struct. Biol., 10:482-488, 2003.
Lin et al., Mol. Cell. Biol., 10:850, 1990.
Luria et al., EMBO J., 6:3307, 1987.
Lusky and Botchan, Proc. Nat'l Acad. Sci. USA., 83:3609, 1986.
Lusky et al., Mol. Cell. Biol., 3:1108, 1983.
MacKenzie and To, J. Immunol. Methods, 220:39, 1998.
MacKenzie et al., J. Biol. Chem., 271:1527, 1996.
Maenaka et al., Biochem Biophys Res Commun., 218:682, 1996.
Majors and Varmus, Proc. Nat'l Acad. Sci. USA., 80:5866, 1983.
Malmborg et al., J. Immunol. Methods, 198:51, 1996.
Marciano et al., Science 284:1516, 1999.
Marks et al., Bio/Technol. 10:779, 1992.
Marks et al., J. Mol. Biol., 222:581, 1991.
Martinez et al., Biochemistry, 35:1179, 1996.
Martinez et al., J. Biotechnol., 71:59, 1999.
Masuda K et al. PNAS 99 (11):7390, 2002.
Maynard et al., Nat. Biotechnol., 20:597-601, 2002.
McNeall et al., Gene, 76:81, 1989.
Miksicek et al., Cell, 46:203, 1986.
Mingarro et al., Trends Biotechnol., 15:432-437, 1997.
Miroux and Walker, J. Mol. Biol., 260:289-298, 1996.
Mitchell et al., Ann. N.Y. Acad. Sci., 690:153, 1993.
Mordacq and Linzer, Genes and Dev., 3:760, 1989.
Moreau et al., Nucl. Acids Res., 9:6047, 1981.
Morrison, et al., Proc. Nat'l. Acad. Sci USA. 81:6851, 1984.
Muesing et al., Cell, 48:691, 1987.
Munson & Pollard, Anal. Biochem. 107:220, 1980.
Mutuberria et al., J. Immunol. Methods, 231:65, 1999.
Nakae, J. Biol. Chem., 251:2176, 1976.
Neu and Heppel, J. Biol. Chem., 240:3685-3692, 1965.
Nikaido and Nakae, Adv. Microb. Physiol., 20:163, 1979.
Nikaido and Vaara, Microbiol. Rev. 49:1, 1985.
Nissim et al., EMBO J., 13:692, 1994.
Nomoto et al., Gene, 236:259, 1999.
Ohara et al., “One-sided polymerase chain reaction: the amplification of cDNA,”
Oka et al, Proc. Natl. Acad. Sci. U.S.A., Vol 82, pp 7212-7216, November 1985
Olsen et al., Nat. Biotechnol, 18:1071-1074, 2000.
O'Shannessy et al., J. Immun. Meth., 99, 153-161, 1987.
Owens & Haley, J. Biol. Chem., 259:14843-14848, 1987.
Painbeni et al., Proc Natl. Acad. Sci. USA, 94:6712, 1997.
Palmiter et al., Nature, 300:611, 1982.
Pech et al., Mol. Cell. Biol., 9:396, 1989.
Perez-Stable and Constantini, Mol. Cell. Biol., 10:1116, 1990.
Picard and Schaffner, Nature, 307:83, 1984.
Pini et al., J. Biol. Chem., 273:21769, 1998.
Pinkert et al., Genes and Dev., 1:268, 1987.
Ponta et al., Proc. Nat'l Acad. Sci. USA., 82:1020, 1985.
Porton et al., Mol. Cell. Biol., 10:1076, 1990.
Potter & Haley, Meth. in Enzymol., 91, 613-633, 1983.
Pugsley, Microbiol. Rev., 57:50-108, 1993.
Queen and Baltimore, Cell, 35:741, 1983.
Quinn et al., Mol. Cell. Biol., 9:4713, 1989.
Rakonjac and Model, J. Mol. Biol., 282:25, 1998.
Rakonjac et al., J. Mol. Biol., 289:1253, 1999.
Rao and Torriani, J. Bacteriol., 170, 5216, 1988.
Redondo et al., Science, 247:1225, 1990.
Reisman and Rotter, Mol. Cell. Biol., 9:3571, 1989.
Resendez Jr. et al., Mol. Cell. Biol., 8:4579, 1988.
Ripe et al., Mol. Cell. Biol., 9:2224, 1989.
Rittling et al., Nucl. Acids Res., 17:1619, 1989.
Rodi and Makowski, Curr. Opin. Biotechnol., 10:87-93, 1999.
Rosen et al., Cell, 41:813, 1988.
Rosovitz et al., J. Biol. Chem 278:30936, 2003.
Sagt et al., Appl. Environ. Microbiol., 68:2155-2160, 2002.
Sakai et al., Genes and Dev., 2:1144, 1988.
Sambrook et al., In: Molecular Cloning: A Laboratory Manual, Vol. 1, Cold Spring Harbor Laboratory Press, Cold Spring Harbor, N.Y., Ch. 7, 7.19-17.29, 1989.
Samuelson et al., Nature, 406:637-641, 2000.
Sasso et al., J. Immunol., 142:2778-2783, 1989.
Satake et al., J. Virology, 62:970, 1988.
Sblattero and Bradbury, Nat. Biotechnol., 18:75, 2000.
Schaffner et al., J. Mol. Biol., 201:81, 1988.
Searle et al., Mol. Cell. Biol., 5:1480, 1985.
Seydel et al., Mol. Microbiol., 34:810-821, 1999.
Sharp and Marciniak, Cell, 59:229, 1989.
Shaul and Ben-Levy, EMBO J., 6:1913, 1987.
Sheets et al., Proc. Natl. Acad. Sci. USA., 95:6157, 1998.
Sherman et al., Mol. Cell. Biol., 9:50, 1989.
Shorki et al., J. Immunol., 146:936-940, 1991.
Shusta et al., J. Mol. Biol., 292:949, 1999.
Silvermann et al., J. Clin. Invest., 96:417-426, 1995.
Sleigh and Lockett, J. EMBO, 4:3831, 1985.
Smith, Science, 228:1315-1317, 1985.
Somerville et al., Appl. Microbiol. Biotechnol., 42:595-603, 1994.
Spalholz et al., Cell, 42:183, 1985.
Spandau and Lee, J. Virology, 62:427, 1988.
Spandidos and Wilkie, EMBO J., 2:1193, 1983.
Stathopoulos et al., Appl. Microbiol. Biotechnol., 45:112-119, 1996.
Stephens and Hentschel, Biochem. J, 248:1, 1987.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
T. J. Gibson, PhD thesis, University of Cambridge (1984).
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Thiesen et al., J Virology, 62:614, 1988.
Thompson et al., J. Mol. Biol. 256, 77, 1999????.
Thorstenson et al., J. Bacteriol., 179:5333, 1997.
Tomlinson et al., J. Mol. Biol. 227:776, 1992.
Tronche et al., Mol. Biol. Med., 7:173, 1990.
Tronche et al., Mol. Cell. Biol., 9:4759, 1989.
Trudel and Constantini, Genes and Dev., 6:954, 1987.
Tsumaki et al., J Biol. Chem. 273:22861, 1998.
Van Wielink and Duine, Trends Biochem Sci., 15:136, 1990.
Vannice and Levinson, J. Virology, 62:1305, 1988.
Vasseur et al., Proc. Natl. Acad. Sci. USA., 77:1068, 1980.
Vaughan et al., Nat. Biotechnol., 14:309, 1996.
Walker et al., Nucleic Acids Res. 20:1691, 1992
Wang and Calame, Cell, 47:241, 1986.
Waterhouse et al., Nucl. Acids Res. 21, 2265-2266 (1993)
Watson, M. Nucleic Acids Research, Vol 12, No. 13, 1984, pp. 5145-5164),
Weber et al., Cell, 36:983, 1984.
Weinberger et al. Mol. Cell. Biol., 8:988, 1984.
Winoto and Baltimore, Cell, 59:649, 1989.
Winter et al, Ann. Rev. Immunol. 12: 433, 1994.
Wittrup, Nat. Biotechnol., 18:1039-1040, 2000.
Wu et al., Biochem Biophys Res Commun. 233:221, 1997.
Yakushi et al., Nat. Cell. Biol., 2:212-218, 2000.
Yakushi T. et al. Journal of Bacteriology 179 (9):2857, 1997.
Yamaguchi and Inouye., Journal of Bacteriology 170 no. 8: 3747, 1988.
Yamaguchi et al., Cell, 53:423-432, 1988.
Yu et al., J. Biol. Chem., 261:2284-2288, 1986.
Yutzey et al. Mol. Cell. Biol., 9:1397, 1989.
Zhao-Emonet et al., Gene Ther. 6:1638, 1999.

What is claimed is:

1. A method of obtaining a bacterium comprising a nucleic acid sequence encoding a binding polypeptide having specific affinity for a target ligand comprising the steps of:
(a) providing a Gram negative bacterium comprising an inner membrane, an outer membrane and a periplasm; said bacterium comprising a nucleic acid sequence encoding a candidate binding polypeptide comprising an inner membrane anchor polypeptide; wherein the bacterium further comprises a nucleic acid sequence encoding a target ligand and wherein the target ligand is exported to the periplasm;
(b) allowing the target ligand to bind to the candidate binding polypeptide in said periplasm;
(c) removing unbound target ligand from said periplasm; and
(d) selecting the bacterium based on the presence of the target ligand bound to the candidate binding polypeptide.

2. The method of claim 1, further defined as a method of obtaining a nucleic acid sequence encoding a binding polypeptide having a specific affinity for a target ligand, the method further comprising the step of:
(d) cloning said nucleic acid sequence encoding a candidate binding polypeptide from said bacterium.

3. The method of claim 1, wherein selecting said bacterium comprises use of a second binding polypeptide having specific affinity for the target ligand to label said target ligand bound to the candidate binding polypeptide.

4. The method of claim 3, wherein the second binding polypeptide is an antibody or fragment thereof.

5. The method of claim 4, wherein the antibody or fragment thereof is fluorescently labeled.

6. The method of claim 3, wherein selecting said bacterium comprises use of at least a third binding polypeptide having specific affinity for the target ligand and/or said second binding polypeptide to label said bacterium.

7. The method of claim 1, wherein the target ligand is fused to a detectable label.

8. The method of claim 7, wherein the detectable label is an antigen.

9. The method of claim 7, wherein the detectable label is GFP.

10. The method of claim 7, wherein the target ligand is further defined as fused to a cytoplasmic degradation signal.

11. The method of claim 10, wherein the cytoplasmic degradation signal is SsrA.

12. The method of claim 1, wherein said Gram negative bacterium is an E. Coli bacterium.

13. The method of claim 1, wherein step (a) is further defined as comprising providing a population of Gram negative bacteria.

14. The method of claim 13, wherein said population of bacteria is defined as collectively expressing nucleic acid sequences encoding a plurality of candidate binding polypeptides.

15. The method of claim 13, wherein said population of bacteria is further defined as collectively expressing nucleic acid sequences encoding a plurality of target ligands.

16. The method of claim 14, wherein the population of bacteria expresses a single target ligand.

17. The method of claim 13, wherein from about two to six rounds of selecting are carried out to obtain said bacterium from said population.

18. The method of claim 2, wherein the bacterium is non-viable.

19. The method of claim 2, wherein the bacterium is viable.

20. The method of claim 2, wherein cloning comprises amplification of the nucleic acid sequence.

21. The method of claim 1, wherein the candidate binding polypeptide is a fusion polypeptide.

22. The method of claim 1, wherein selecting is carried out by flow-cytometry or magnetic separation.

23. The method of claim 1, wherein said candidate binding polypeptide is further defined as an antibody or fragment thereof.

24. The method of claim 23, wherein said candidate binding polypeptide is further defined as a scAb, Fab or scFv.

25. The method of claim 1, wherein said candidate binding polypeptide is further defined as an enzyme.

26. The method of claim 1, wherein said target ligand is selected from the group consisting of a peptide, a polypeptide, an enzyme, a nucleic acid and a small molecule.

27. The method of claim 1, wherein said nucleic acid encoding a candidate binding polypeptide is flanked by known PCR primer sites.

28. The method of claim 1, wherein step (c) comprises permeabilizing and/or removing said outer membrane.

29. The method of claim 28, wherein permeabilizing and/or removing the outer membrane comprises a method selected from the group consisting of: treatment with hyperosmotic conditions, treatment with physical stress, infecting the bacterium with a phage, treatment with lysozyme, treatment with EDTA, treatment with a digestive enzyme and treatment with a chemical that disrupts the outer membrane.

30. The method of claim 28, comprising removing the outer membrane.

31. The method of claim 29, wherein permeabilizing and/or removing the outer membrane comprises a combination of said methods.

32. The method of claim 31, wherein permeabilizing and/or removing the outer membrane comprises treatment with lysozyme and EDTA.

33. The method of claim 28, wherein permeabilizing and/or removing the outer membrane comprises treating the bacterium with a combination of physical, chemical and enzyme disruption of the outer membrane.

34. The method of claim 28, wherein said bacterium comprises a mutation conferring increased permeability of said outer membrane.

35. The method of claim 1, wherein step (c) comprises permeabilizing the outer membrane and washing the cell.

36. The method of claim 1, wherein said bacterium is grown at a sub-physiological temperature.

37. The method of claim 36, wherein said sub-physiological temperature is about 25° C.

38. The method of claim 1, wherein said target ligand and said candidate binding polypeptide are reversibly bound.

39. The method of claim 1, wherein the target ligand is operably linked to a leader sequence capable of directing the export of the target ligand to the periplasm.

40. The method of claim 39, wherein the leader peptide is an ssTorA leader peptide.

41. The method of claim 1, wherein said inner membrane anchor polypeptide comprises a transmembrane protein or fragment thereof.

42. The method of claim 41, wherein the transmembrane protein or fragment thereof comprises a sequence selected from the group consisting of: the first two amino acids encoded by the E. coli NlpA gene, the first six amino acids encoded by the E. coli NlpA gene, the gene III protein of filamentous phage or a fragment thereof, an inner membrane lipoprotein or fragment thereof.

43. The method of claim 41, wherein the inner membrane anchor polypeptide is fused to the candidate binding polypeptide via an N- or C-terminus.

44. The method of claim 1, wherein the inner membrane anchor polypeptide comprises an inner membrane lipoprotein or fragment thereof selected from the group consisting of: AraH, MglC, MalF, MalG, Mal C, MalD, RbsC, RbsC, ArtM, ArtQ, GlnP, ProW, HisM, H is Q, LivH, LivM, LivA, Liv E,Dpp B, DppC, OppB,AmiC, AmiD, BtuC, FhuB, FecC, FecD,FecR, FepD, NikB, NikC, CysT, CysW, UgpA, UgpE, PstA, PstC, PotB, PotC,PotH, PotI, ModB, NosY, PhnM, LacY, SecY, TolC, DsbB, DsbD, TonB, TatC, CheY, TraB, Exb D, ExbB and Aas.

45. A method of obtaining a bacterium comprising a nucleic acid sequence encoding a binding polypeptide having specific affinity for a target ligand comprising the steps of:
(a) providing a Gram negative bacterium comprising an inner membrane, an outer membrane and a periplasm; said bacterium comprising a nucleic acid sequence encoding a candidate binding polypeptide, wherein the candidate binding polypeptide is anchored to the outer side of the inner membrane with an inner membrane anchor polypeptide; wherein the bacterium further comprises a nucleic acid sequence encoding a target ligand, wherein the target ligand is exported to the periplasm;
(b) allowing the target ligand to bind to the candidate binding polypeptide;
(c) removing the outer membrane of said bacterium; and
(c) selecting the bacterium based on the presence of the target ligand bound to the candidate binding polypeptide on the outer side of the inner membrane.

46. A method of obtaining a bacterium comprising a nucleic acid sequence encoding a binding polypeptide having specific affinity for a target ligand comprising the steps of:
(a) providing a population of Gram negative bacteria the members of which comprise an inner membrane, an outer membrane and a periplasm; said population collectively comprising nucleic acid sequences encoding plurality of candidate binding polypeptides, wherein the candidate binding polypeptides are anchored to the outer side of the inner membrane of said bacteria; wherein the bacteria further comprise nucleic acid sequences encoding a target ligand, wherein the target ligand is exported to the periplasm;
(b) allowing the target ligand to bind to the candidate binding protein in said periplasm;
(c) removing the outer membrane of said bacterium; and
(d) selecting the bacterium from said population based on the presence of the target ligand bound to the candidate binding polypeptide on the outer side of the inner membrane.

47. The method of claim 46, wherein step (d) is further defined as selecting a subpopulation of bacteria comprising the target ligand bound to the candidate binding polypeptide.

48. The method of claim 46, wherein step (d) comprises fluorescently labeling said target ligand followed by fluorescence activated cell sorting (FACS).

49. A method of obtaining a bacterium comprising a nucleic acid sequence encoding a binding polypeptide having specific affinity for a target ligand comprising the steps of:
(a) providing a Gram negative bacterium comprising an inner membrane, an outer membrane and a periplasm; said bacterium comprising a nucleic acid sequence encoding a candidate binding polypeptide, wherein the candidate binding polypeptide is anchored to the outer side of the inner membrane; wherein the bacterium further comprises a nucleic acid sequence encoding a fusion polypeptide comprising a target ligand, a periplasmic export signal, a fluorescent label and a cytoplasmic degradation signal;
(b) allowing the target ligand to bind to the candidate binding polypeptide;
(c) removing the outer membrane of said bacterium; and
(d) selecting the bacterium based on the presence of the target ligand bound to the candidate binding polypeptide on the outer side of the inner membrane using fluorescence activated cell sorting (FACS).

50. The method of claim 49, wherein the periplasmic export signal is TorA.

51. The method of claim 49, wherein the cytoplasmic degradation signal is SsrA.

52. The method of claim 49, wherein the fluorescent label is GFP.

53. The method of claim 49, wherein the fusion polypeptide comprises the following components from the N-terminus to C-terminus: a periplasmic export signal, a target ligand, a fluorescent label and a cytoplasmic degradation signal.

54. The method of claim 7, wherein the detectable label comprises the peptide sequence of SEQ ID NO:33.


SEQUENCE LISTING (59 sequences)

SEQ ID NO: 1 - DNA, 17 bp, Artificial Sequence
  Description of Artificial Sequence  Synthetic Primer
caggaaacagctatgac

SEQ ID NO: 2 - DNA, 17 bp, Artificial Sequence
  Description of Artificial Sequence  Synthetic Primer
gaattttctgtatgagg

SEQ ID NO: 3 - DNA, 18 bp, Artificial Sequence
  Description of Artificial Sequence  Synthetic Primer
gccacctccgcctgaacc

SEQ ID NO: 4 - DNA, 17 bp, Artificial Sequence
  Description of Artificial Sequence  Synthetic Primer
ctatgcggccccattca

SEQ ID NO: 5 - DNA, 5 bp, Artificial Sequence
  Description of Artificial Sequence  Synthetic Primer
aaaaa

SEQ ID NO: 6 - DNA, 39 bp, Artificial Sequence
  Description of Artificial Sequence  Synthetic Primer
gaaggagatatacatatgaaactgacaacacatcatcta

SEQ ID NO: 7 - DNA, 45 bp, Artificial Sequence
  Description of Artificial Sequence  Synthetic Primer
ctgggccatggccggctgggcctcgctgctactctggtcgcaacc

SEQ ID NO: 8 - protein, 7 aa, Artificial Sequence
  Description of Artificial Sequence  Synthetic Peptide
QTTHVPP

SEQ ID NO: 9 - protein, 7 aa, Artificial Sequence
  Description of Artificial Sequence  Synthetic Peptide
QTTHVPP

SEQ ID NO: 10 - protein, 7 aa, Artificial Sequence
  Description of Artificial Sequence  Synthetic Peptide
QTTHSPA

SEQ ID NO: 11 - protein, 7 aa, Artificial Sequence
  Description of Artificial Sequence  Synthetic Peptide
QTTHLPT

SEQ ID NO: 12 - protein, 7 aa, Artificial Sequence
  Description of Artificial Sequence  Synthetic Peptide
QTTHTPP

SEQ ID NO: 13 - protein, 7 aa, Artificial Sequence
  Description of Artificial Sequence  Synthetic Peptide
QTTHTPP

SEQ ID NO: 14 - protein, 7 aa, Artificial Sequence
  Description of Artificial Sequence  Synthetic Peptide
QTTHIPT

SEQ ID NO: 15 - protein, 7 aa, Artificial Sequence
  Description of Artificial Sequence  Synthetic Peptide
QTTHVPP

SEQ ID NO: 16 - protein, 7 aa, Artificial Sequence
  Description of Artificial Sequence  Synthetic Peptide
QTTHVPA

SEQ ID NO: 17 - protein, 7 aa, Artificial Sequence
  Description of Artificial Sequence  Synthetic Peptide
QTTHIPA

SEQ ID NO: 18 - protein, 7 aa, Artificial Sequence
  Description of Artificial Sequence  Synthetic Peptide
QTTHLPA

SEQ ID NO: 19 - protein, 7 aa, Artificial Sequence
  Description of Artificial Sequence  Synthetic Peptide
QTTHVPC

SEQ ID NO: 20 - DNA, 741 bp, Artificial Sequence
  Description of Artificial Sequence  Synthetic Primer
gatattcagatgacacagactacatcctccctgtctgcctctctgggagacagagtcaccatcagttgcagggcaagtcaggacattaggaattatttaaactggtatcagcagaaaccagatggaactgttaaactcctgatctactacacatcaagattacagtcaggagtcccatcaaggttcagtggcagtgggtctggaacagattattctctcaccattagcaaccaggagcaagaagatattggcacttacttttgccaacagggtaatacgcttccgtggacgttcggtggaggcaccaagctggaaataaaacgtggtggtggtggttctggtggtggtggttctggcggcggcggctccggtggtggtggatccgaggtccaactgcaacagtctggacctgagctggtgaagcctggggcctcagtgaagatttcctgcaaagattctggctacgcattcagtagctcttggatgaactgggtgaagcagaggcctggacagggtcttgagtggattggacggatttatcctggagatggagatactaactacaatgggaagttcaagggcaaggccacactgactgcagacaaatcctccagcacagcctacatgcagctcagcagcctgacctctgtggactctgcggtctatttctgtgcaagatcggggttactacgttatgctatggactactggggtcaaggaacctcagtcaccgtctcctcg

SEQ ID NO: 21 - protein, 247 aa, Artificial Sequence
  Description of Artificial Sequence  Synthetic Peptide
DIQMTQTTSSLSASLGDRVTISCRASQDIRNYLNWYQQKPDGTVKLLIYYTSRLQSGVPSRFSGSGSGTDYSLTISNQEQEDIGTYFCQQGNTLPWTFGGGTKLEIKRGGGGSGGGGSGGGGSGGGGSEVQLQQSGPELVKPGASVKISCKDSGYAFSSSWMNWVKQRPGQGLEWIGRIYPGDGDTNYNGKFKGKATLTADKSSSTAYMQLSSLTSVDSAVYFCARSGLLRYAMDYWGQGTSVTVSS

SEQ ID NO: 22 - DNA, 741 bp, Artificial Sequence
  Description of Artificial Sequence  Synthetic Primer
gatattcagatgacacagactacatcctccctgtctgcctctctgggagacagagtcaccgtcagttgcagggcaagtcaggacattaggaattatttaaactggtatcagcagaaaccagacggaactgttaaattcctgatctactacacatcaagattacagccaggagtcccatcaaggttcagtggcagtgggtctggaacagattattccctcaccattaacaacctggagcaggaagatattggcacttacttttgccaacagggcaatacgcctccgtggacgttcggtggaggcaccaagctggaaataaaacgtggtggaggtggttctgatggtggtggttctggcggcggcggctccggtggtggtggatccgaggtccaactgcaacagtctggacctgagctggtgaagcctggggcctcagtgaagatttcctgcaaagattctggctacgcattcaatagctcttggatgaactgggtgaagcagaggcctggacagggtcttgagtggattggacggatttatcctggagatggagattctaactacaatgggaaattcgagggcaaggccatactgactgcagacaaatcctccagcacagcctacatgcagctcagcagcctgacctctgtggactctgcggtctatttctgtgcaagatcggggttgctacgttatgctatggactactggggtcaaggaacctcagtcaccgtctcctcg

SEQ ID NO: 23 - protein, 247 aa, Artificial Sequence
  Description of Artificial Sequence   Synthetic Peptide
DIQMTQTTSSLSASLGDRVTVSCRASQDIRNYLNWYQQKPDGTVKFLIYYTSRLQPGVPSRFSGSGSGTDYSLTINNLEQEDIGTYFCQQGNTPPWTFGGGTKLEIKRGGGGSDGGGSGGGGSGGGGSEVQLQQSGPELVKPGASVKISCKDSGYAFNSSWMNWVKQRPGQGLEWIGRIYPGDGDSNYNGKFEGKAILTADKSSSTAYMQLSSLTSVDSAVYFCARSGLLRYAMDYWGQGTSVTVSS

SEQ ID NO: 24 - DNA, 741 bp, Artificial Sequence
  Description of Artificial Sequence  Synthetic Primer
gatattcagatgacacagactacatcctccctgtctgcctctctgggagacagagtcaccgtcagttgcagggcaagtcaggacattaggaattatttaaactggtatcagcagaaaccagacggaactgttaaattcctgatctactacacatcaagattactgccaggagtcccatcaaggttcagtggcagtgggtctggaacagattattccctcaccattaacaacctggagcaggaagatattggcacttacttttgccaacagggcaatacgcctccgtggacgttcggtggaggcaccaagctggaaataaaacgtggtggaggtggttctgatggtggtggttctggcggcggcggctccggtggtggtggatccgaggtccaactgcaacagtctggacctgagctggtgaagcctggggcctcagtgaagatttcctgcaaagattctggctacgcattcaatagctcttggatgaactgggtgaagcagaggcctggacagggtcttgagtggattggacggatttatcctggagatggagattctaactacaatgggaaattcgagggcaaggccatactgacagcagacaaatcctccagcacagcctacatgcagctcagcagcctgacctctgtggactctgcggtctatttctgtgcaagatcggggttgctacgttatgctatggactactggggtcaaggaacctcagtcaccgtctcctcg

SEQ ID NO: 25 - protein, 247 aa, Artificial Sequence
  Description of Artificial Sequence  Synthetic Peptide
DIQMTQTTSSLSASLGDRVTVSCRASQDIRNYLNWYQQKPDGTVKFLIYYTSRLLPGVPSRFSGSGSGTDYSLTINNLEQEDIGTYFCQQGNTPPWTFGGGTKLEIKRGGGGSDGGGSGGGGSGGGGSEVQLQQSGPELVKPGASVKISCKDSGYAFNSSWMNWVKQRPGQGLEWIGRIYPGDGDSNYNGKFEGKAILTADKSSSTAYMQLSSLTSVDSAVYFCARSGLLRYAMDYWGQGTSVTVSS

SEQ ID NO: 26 - protein, 6 aa, Escherichia coli
CDQSSS

SEQ ID NO: 27 - DNA, 19 bp, Artificial Sequence
  Description of Artificial Sequence  Synthetic Primer
agcggatcctacctgacgc

SEQ ID NO: 28 - DNA, 37 bp, Artificial Sequence
  Description of Artificial Sequence  Synthetic Primer
ccttgaaggtgaaacaagcgtcagtcgccgcttgcgc

SEQ ID NO: 29 - DNA, 40 bp, Artificial Sequence
  Description of Artificial Sequence  Synthetic Primer
gttcggattgttttgaaattccttgaaggtgaaacaagcg

SEQ ID NO: 30 - DNA, 40 bp, Artificial Sequence
  Description of Artificial Sequence  Synthetic Primer
ctttaccagagaacgcgggttcggattgttttgaaattcc

SEQ ID NO: 31 - DNA, 33 bp, Artificial Sequence
  Description of Artificial Sequence  Synthetic Primer
cgtctagatccaccctttaccagagaacgcggg

SEQ ID NO: 32 - protein, 18 aa, Artificial Sequence
  Description of Artificial Sequence  Synthetic Peptide
CFTFKEFQNNPNPRSLVK

SEQ ID NO: 33 - protein, 8 aa, Artificial Sequence
  Description of Artificial Sequence  Synthetic Peptide
DYKDDDDK

SEQ ID NO: 34 - DNA, 30 bp, Artificial Sequence
  Description of Artificial Sequence  Synthetic Primer
gtcgagctcagagaaggagatatacatatg

SEQ ID NO: 35 - DNA, 48 bp, Artificial Sequence
  Description of Artificial Sequence  Synthetic Primer
ctttgtcatcgtcatctttataatctggtgcagcggccgcgaattcgg

SEQ ID NO: 36 - DNA, 43 bp, Artificial Sequence
  Description of Artificial Sequence  Synthetic Primer
cgaagcttctattaggcgcgccctttgtcatcgtcatctttat

SEQ ID NO: 37 - DNA, 567 bp, Artificial Sequence
  Description of Artificial Sequence  Synthetic Primer
atgaaatccctattgcctacggcagccgctggattgttattactcgcggcccagccggccatggcgaaacgcttccactacgatcgtaacaatatcgcggtgggtgccgatgaaagcgtggttaaagaagcgcaccgcgaagtgatcaactctagcaccgagggcctgctgttgaacattgataaagacatccgtaagattctgagcggttacatcgtggaaattgaagacaccgagggcttgaaagaagttatcaacgatcgctatgacatgctgaatatctccagcctgcgccaggatggtaaaaccttcattgatttcaaaaagtacaacgacaaattgccgctggcgatcagcaatccaaactacgaagtgaacgtttacgcagtgacgaaagaaaataccatcattaacccatctgagaacggcgatacctccaccaatggtatcaagaaaatcctgatttttagcaaaaagggctatgaaattggttcggcctcgggggccgaattcgcggccgctgcaccagattataaagatgacgatgacaaagggcgcgcctaatag

SEQ ID NO: 38 - protein, 187 aa, Artificial Sequence
  Description of Artificial Sequence  Synthetic Peptide
MKSLLPTAAAGLLLLAAQPAMAKRFHYDRNNIAVGADESVVKEAHREVINSSTEGLLLNIDKDIRKILSGYIVEIEDTEGLKEVINDRYDMLNISSLRQDGKTFIDFKKYNDKLPLAISNPNYEVNVYAVTKENTIINPSENGDTSTNGIKKILIFSKKGYEIGSASGAEFAAAAPDYKDDDDKGRA

SEQ ID NO: 39 - DNA, 50 bp, Artificial Sequence
  Description of Artificial Sequence  Synthetic Primer
gatcgctatgacatgctgaatatctccagcctgcgccaggatggtaaaac

SEQ ID NO: 40 - DNA, 49 bp, Artificial Sequence
  Description of Artificial Sequence  Synthetic Primer
agacaccgagggcttgaaagaagttatcaacgatcgctatgacatgctg

SEQ ID NO: 41 - DNA, 49 bp, Artificial Sequence
  Description of Artificial Sequence  Synthetic Primer
gtaagattctgagcggttacatcgtggaaattgaagacaccgagggctt

SEQ ID NO: 42 - DNA, 50 bp, Artificial Sequence
  Description of Artificial Sequence  Synthetic Primer
ggcctgctgttgaacattgataaagacatccgtaagattctgagcggtta

SEQ ID NO: 43 - DNA, 50 bp, Artificial Sequence
  Description of Artificial Sequence  Synthetic Primer
cgcaccgcgaagtgatcaactctagcaccgagggcctgctgttgaacatt

SEQ ID NO: 44 - DNA, 49 bp, Artificial Sequence
  Description of Artificial Sequence  Synthetic Primer
gtgggtgccgatgaaagcgtggttaaagaagcgcaccgcgaagtgatca

SEQ ID NO: 45 - DNA, 50 bp, Artificial Sequence
  Description of Artificial Sequence  Synthetic Primer
aaacgcttccactacgatcgtaacaatatcgcggtgggtgccgatgaaag

SEQ ID NO: 46 - DNA, 42 bp, Artificial Sequence
  Description of Artificial Sequence  Synthetic Primer
gctaggcccagccggccatggcgaaacgcttccactacgatc

SEQ ID NO: 47 - DNA, 49 bp, Artificial Sequence
  Description of Artificial Sequence  Synthetic Primer
tttgtcgttgtactttttgaaatcaatgaaggttttaccatcctggcgc

SEQ ID NO: 48 - DNA, 49 bp, Artificial Sequence
  Description of Artificial Sequence  Synthetic Primer
tagtttggattgctgatatacagcggcaatttgtcgttgtactttttga

SEQ ID NO: 49 - DNA, 50 bp, Artificial Sequence
  Description of Artificial Sequence  Synthetic Primer
ttctttcgtcactgcgtaaacgttcactttgtagtttggattgctgatat

SEQ ID NO: 50 - DNA, 49 bp, Artificial Sequence
  Description of Artificial Sequence  Synthetic Primer
gccgttctcagatgggttaatgatggtattttctttcgtcactgcgtaa

SEQ ID NO: 51 - DNA, 49 bp, Artificial Sequence
  Description of Artificial Sequence  Synthetic Primer
caggattttcttgataccattggtggaggtatcgccgttctcagatggg

SEQ ID NO: 52 - DNA, 50 bp, Artificial Sequence
  Description of Artificial Sequence  Synthetic Primer
accaatttcatagccctttttgctaaaaatcaggattttcttgataccat

SEQ ID NO: 53 - DNA, 42 bp, Artificial Sequence
  Description of Artificial Sequence  Synthetic Primer
gctaggcccccgaggccgaaccaatttcatagccctttttgc

SEQ ID NO: 54 - DNA, 29 bp, Artificial Sequence
  Description of Artificial Sequence  Synthetic Primer
caaaaaggcgaacgacaaattgccgctgt

SEQ ID NO: 55 - DNA, 38 bp, Artificial Sequence
  Description of Artificial Sequence  Synthetic Primer
caatttgtcgttcgcctttttgaaatcaatgaaggttt

SEQ ID NO: 56 - DNA, 34 bp, Artificial Sequence
  Description of Artificial Sequence  Synthetic Primer
ttgccgctggcgatcagcaatccaaactacaaag

SEQ ID NO: 57 - DNA, 28 bp, Artificial Sequence
  Description of Artificial Sequence  Synthetic Primer
gctgatcgccagcggcaatttgtcgttg

SEQ ID NO: 58 - DNA, 34 bp, Artificial Sequence
  Description of Artificial Sequence  Synthetic Primer
gcaagcttagagaaggagatatacatatgaaatc

SEQ ID NO: 59 - DNA, 27 bp, Artificial Sequence
  Description of Artificial Sequence  Synthetic Primer
ccaagcttctattaggcgcgccctttg